(19) United States
(12) Patent Application Publication
(10) Pub. No.: US 2004/0259129 A1
(43) Pub. Date: Dec. 23, 2004

(54) Compositions and methods for identifying genes whose products modulate biological processes

(76) Inventors: Eugene S. Kandel, Beachwood, OH (US); George R. Stark, Shaker Heights, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/806,040

(22) Filed: Mar. 22, 2004

Related U.S. Application Data
(60) Provisional application No. 60/456,321, filed on Mar. 20, 2003.

(51) Int. Cl. C12Q 1/68; C12N 15/74

(52) U.S. Cl. 435/6; 435/473

(57) ABSTRACT

Methods of identifying a gene whose product modulates a control phenotype of interest are provided. The methods comprise introducing a promoter insertion construct of the present invention into the genomes of a collection of host cells having the control phenotype of interest; selecting mutagenized cells exhibiting a mutant phenotype to provide a pool of mutant cells; treating the mutant cells with the disrupting agent having recombinase activity; detecting changes or the lack thereof in the linkage between the promoter element and downstream host genomic DNA sequences in treated mutant cells, or in both treated and untreated mutant cells; and correlating the changes or lack thereof in the linkage between the promoter element and the downstream host genomic DNA sequences in treated mutant cells, or in both untreated and treated mutant cells, with the phenotypes of said cells. Also provided are compositions that are used in the present methods.

### CROSS-REFERENCE TO RELATED APPLICATION

This invention claims priority to U.S. Provisional Patent Application Ser. No. 60/456,321, filed Mar. 20, 2003, which is incorporated herein in its entirety.

### STATEMENT ON GOVERNMENT FUNDED RESEARCH

The present invention was made, at least in part, with support from the National Institute of General Medical Sciences Grant RO1 GM 049345. The United States Government has certain rights in the invention.

### FIELD OF THE INVENTION

The present invention relates to compositions and methods for easily and reliably identifying genes whose products modulate biological processes in eukaryotic cells, particularly in mammalian cells.

### BACKGROUND OF THE INVENTION

There have been numerous attempts to isolate and identify genes involved in various biological processes. One commonly-used method involves expression selection of nucleic acids from appropriately engineered libraries (such as full-length cDNA libraries, or libraries of truncated randomly oriented cDNA fragments called GSEs) (Murphy A J, Efstratiadis A. “Cloning vectors for expression of cDNA libraries in mammalian cells” Proc. Natl. Acad. Sci. U.S.A., 1987 Dec. 84(23):8277-81; Deiss L P, Mimchi A. “A genetic tool used to identify thioredoxin as a mediator of a growth inhibitory signal” Science, 1991 Apr. 5, 252(5002):117-20; Roninson I B, Gudkov A V, Holzmayer T A, Krischling D J, Kazarov A R, Zelnick C R, Mazo I A, Axenovich S, Thimmapaya R., “Genetic suppressor elements: new tools for molecular oncology” Thirteenth Cornelius Rhoads Memorial Awards Lecture, Cancer Res. 1995 Sept. 15, 55(18):4023-8,). Another method involves random integration of DNA fragments throughout a host cell's genome, a process referred to hereinafter as “insertional mutagenesis”. (e.g. Friedrich G, Soriano P., “Promoter traps in embryonic stem cells: a genetic screen to identify and mutate developmental genes in mice” Genes Dev, 1991 September; 5(9):1513-23). Both of these methods serve to generate a genetically diverse population of cells (either in an organism or in tissue culture) from which mutant cells with desired properties or phenotypes are isolated.

The expression selection method involves delivery and expression of the nucleic acids in the library to a collection of host cells and selection of those cells that exhibit a mutant phenotype of interest. A causal relationship between the exogenous nucleic acid that has been delivered to the cell and the mutant phenotype can be validated either by shutting down expression of the exogenous nucleic acid and observing an alteration in the phenotype of interest, or by transferring the expressed nucleic into naive cells and observing a transformation in the cells from the control phenotype to the mutant phenotype of interest. However, the task of constructing and delivering a complete library of all possible cDNA or GSEs is not attainable at this time. Moreover, there are many indications that even the projects that were considered successful failed to identify a great number of factors which, indeed, are involved in the biological processes being studied.

Insertional mutagenesis in its simplest form involves random integration of DNA fragments throughout a host cell's genome and, hence, could disrupt any gene without principal limitations. Insertional mutagenesis also does not require elaborate libraries. In addition, the same vector (either a plasmid or, more commonly, a transposon or a retrovirus) could be used to deliver the disruptive DNA fragment into different cells and even into different organisms. However, simple disruption of a single allele in a diploid cell is usually a recessive event and is not associated with a detectable phenotype. Moreover, the exact integration event can not be reproduced in a naive cell, making it difficult to prove that the insert is the cause of the phenotypic alteration.

Accordingly, it is desirable to have new methods and systems for reliably and efficiently identifying and isolating genes whose products are modulators of biological processes in eukaryotic cells. Methods which can involve steps that can be automated are particularly desirable.

### SUMMARY OF THE INVENTION

The present invention provides methods for reliably and easily identifying genes whose products are modulators of biological processes in a eukaryotic cell. Examples of such genes include, but are not limited to, genes that encode transcription factors, genes that encode enzymes, genes that encode co-factors of known transcription factors (e.g. chromatin modulating enzymes), and genes that encode transporter molecules. The methods comprise introducing a promoter insertion construct of the present invention into the genomes of a collection of host cells having a predetermined, i.e., control, phenotype to provide a population of mutagenized cells; selecting mutagenized cells exhibiting an altered, i.e., mutant, phenotype of interest (hereinafter referred to as “mutant” cells); treating the mutant cells with a disrupting agent having site-specific recombinase activity; characterizing the phenotype of the treated cells; and correlating the phenotypes of the treated cells or the phenotypes of both the untreated and treated mutant cells with changes or lack thereof in the status of the linkage between the promoter element of the promoter insertion construct and genomic DNA sequences, particularly genomic DNA coding sequences, that are downstream of the promoter element in the untreated mutant cells. Changes that can occur as a result of treatment with the disrupting agent include loss of the promoter element and elimination of the linkage between the promoter element and the downstream genomic DNA sequences, or an inversion of the promoter element with respect to the downstream genomic DNA sequences, or a disruption of the linkage between the promoter element and downstream genomic DNA sequences due to insertion of additional nucleotides, e.g., a marker gene, between the promoter element and the downstream DNA sequences. For convenience the disrupting agents that have site-specific recombinase activity are referred to hereinafter as a “recombinase”. If the downstream genomic DNA coding sequence

i) is operably linked with the promoter element of the promoter insertion construct in mutagenized cells that display the mutant phenotype, but is not operably linked with the promoter element in treated cells that display the control phenotype; or

ii) is operably linked with the promoter element of the promoter insertion construct in treated cells that display the mutant phenotype, but is not operably linked with the promoter element in treated cells that display the control phenotype, or

iii) both i and ii;

the downstream genomic DNA coding sequence (referred to hereinafter as the “validated target”) encodes a product that is directly or indirectly involved in modulating a biological process associated with the control phenotype. Preferably, multiple promoter insertion constructs of the present invention are inserted into the genome of each host cell to enhance efficiency of the present method. To enhance the efficiency of the present method, it is also desirable to select mutant cells into whose genome the promoter insertion construct has integrated prior to treating such mutant cells with the disrupting agent. Such selection can be achieved by incorporating a marker gene into the promoter insertion construct.

Depending upon the nature of the control phenotype, the validated target is a host genomic sequence whose operable linkage with the promoter insertion construct is disrupted or inactivated in treated cells that exhibit a control phenotype, or a host genomic sequence whose operable linkage with the promoter insertion construct has not been disrupted in treated cells that maintain a mutant phenotype, or both. In accordance with the present method, integration of the DNA construct into the host cell is not targeted. Thus, the present methods enable isolation and identification of endogenous genes, including those associated with human disease and development, without prior knowledge of the sequence, structure, function, or expression profile of these genes.

In one embodiment, the promoter insertion construct comprises a promoter element that is flanked by a recognition motif for a site specific recombinase (hereinafter referred to as a “recombinase recognition site”) and a downstream recombinase recognition site sequence. In an alternative embodiment, the promoter insertion construct comprises a promoter element and a downstream recombinase recognition site sequence. Such construct lacks an upstream recombinase recognition site sequence. In a preferred embodiment, the promoter insertion construct comprises one or more marker genes or promoterless marker genes for identifying or selecting recombinant host cells. The marker gene may be located upstream of the promoter and downstream of the upstream recombinase recognition sequences. In cases where the marker gene is upstream of the promoter and downstream of the upstream recombinase recognition site, the marker gene, preferably, is operably independent of the promoter element. The marker gene may be downstream of the promoter, either between the promoter element and downstream recombinase recognition site sequence, or downstream of both the promoter element and the downstream recombinase recognition site sequence. In such construct, the marker gene lacks a promoter and is operably linked to the promoter of the promoter insertion construct. In such, construct, an internal ribosome entry site is engineered downstream from the marker gene. Preferably, such construct also comprises a splice donor site downstream from the internal ribosome entry site. In a further embodiment, the construct comprises a promoterless marker gene upstream of the promoter element and the upstream recombinase recognition site. In all embodiments, the promoter insertion construct lacks a transcription terminator downstream of the promoter element.

In one embodiment, the host cells comprise a selection system for selecting mutagenized cells having an altered or mutant phenotype. The selection system comprises a promoter of a gene associated with the control phenotype operably linked to a marker gene. Preferably the promoter is operably linked with a positive selectable marker gene, or a negative selectable marker gene, or to both a positive selectable marker gene and a negative selectable marker gene.

The present invention also relates to promoter insertion constructs used in the present methods and to mutagenized cells, particularly mutagenized cells exhibiting a mutant phenotype, produced in accordance with the present methods.

It is to be understood that both the foregoing general description and the following detailed description are exemplary and explanatory only and are not restrictive of the invention, as claimed.

The accompanying drawings, which are incorporated in and constitute a part of this specification, illustrate embodiments of the invention and together with the description, serve to explain the principles of the invention.

### BRIEF DESCRIPTION OF THE FIGURES

FIG. 1 depicts three possible outcomes that can result from integration of a promoter insertion construct of the present invention into a host cell's genome. When a cellular gene (A) is targeted by a promoter insertion construct (B) the consequences may include production of a full-length (C) or truncated (D) peptide or an anit-sense mRNA (E), depending on the actual integration site and the structure of the gene.

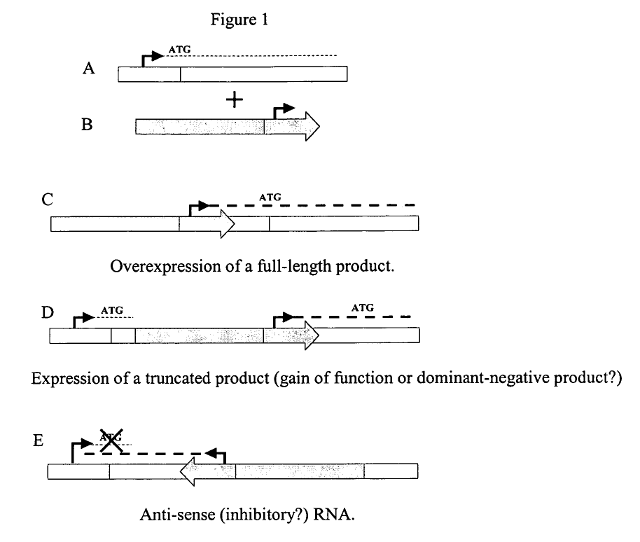

FIG. 2 depicts one example of a promoter insertion construct of the present invention. In such construct, the operational linkage between the promoter and downstream host DNA sequences is disrupted by a recombinase that causes inversion of the fragment flanked by the recognition sites. The ability of the inverted promoter to drive expression in the new direction, following inversion, is curtailed by appropriately placed termination signals.

FIG. 3 depicts one example of a promoter insertion construct of the present invention in which a promoterless marker gene is located upstream of the upstream recognition site. In such construct, the operational linkage between the promoter and downstream host DNA sequences is disrupted by a recombinase that causes inversion of the fragment flanked by the recognition sites. Such inversion establishes a link between the promoter and otherwise promoterless marker gene.

FIG. 4 depicts one example of a promoter insertion construct of the present invention in which the operational linkage between the promoter and downstream host DNA sequences is disrupted by inversion of the fragment lying between the recognition sites. This construct comprises a downstream marker gene operably linked to the promoter and other sequence such an internal ribosome entry site and an uncoupled splice donor site that improve operable linkages between the downstream host DNA and the promoter. Upon inversion the operational linkage between the promoter and the host DNA, as well as the marker, is lost. This result could be monitored by the loss of expression of the marker gene.

FIG. 5 depicts one example of a promoter insertion construct of the present invention in which the operational linkage between the promoter and downstream host DNA sequences is disrupted by removal or excision of the DNA fragment that is flanked by the recognition sites.

FIG. 6 depicts one example of a promoter insertion construct of the present invention that comprises a marker gene operably linked to the promoter and sequences, such as an internal ribosome entry site, and an uncoupled splice donor site that improve operable linkages between the promoter and downstream host DNA. Upon excision of the promoter/marker cassette, the operational linkage between the promoter and the downstream host DNA is lost. This result could be monitored by the loss of expression of the marker gene.

FIG. 7 depicts one example of a promoter insertion construct of the present invention in which the operational linkage between the promoter and the downstream host DNA is inactivated by expression of a recombinase simultaneously with transfection of a plasmid containing a recombinase recognition site. With some probability, the plasmid integrates downstream of the promoter and disrupts the operational linkage between the promoter and the downstream host DNA.

FIG. 8 depicts another embodiment of the promoter insertion construct shown in FIG. 7. In this embodiment, the disrupting plasmid carries a marker gene that becomes functional upon disrupting the promoter-host DNA linkage. The transcript terminus is determined by the signals within the “disrupter” plasmid. A subsequent recombination step restores the operational linkage between the promoter and the host DNA and removes the disrupter fragment from the genome, a result that could be monitored by loss of expression of the marker gene.

FIG. 9 depicts another embodiment of the promoter insertion construct shown in FIG. 7. This construct comprises a marker gene operably linked to the promoter and sequences, such as an internal ribosome entry site, and an uncoupled splice donor site that improve operable linkages between the promoter and downstream host DNA. Upon insertion of the disrupter fragment into the construct, the operational linkage between the promoter and the host DNA is lost, a result that could be monitored by loss of expression of the marker gene.

FIG. 10 shows a construct delivered in the form of a self-inactivation retroviral vector and carrying recognition sites for Cre recombinase (loxP) in its LTRs. “Mini-exon” refers to a fragment comprising a translation start site, an open reading frame, and an unpaired splice donor site. Cre-driven recombination results in the loss of the fragment between the two loxP sites and loss of the operational linkage between the promoter and the downstream host DNA. The presence of the constrcut in a given cell could be determined by the expression of the marker gene that may be expressed independently of the mini-exon.

FIG. 11 is a schematic representation showing how the present method allows identification of genes whose products modulate the phenotype of interest and reduces the findings of false positives.

FIG. 12 is a schematic of an inverse PCR procedure for obtaining inserts that are present in the genome of mutagenized cells that have or have not been treated with a disruption agent.

FIG. 13 is a graph showing the enhanced yield of GCV-resistant clones after infection of HCT9E cells with a transcriptionally-competent retroviral vector. GCV-resistant colonies were counted on 10 plates of mock-infected cells (circles) or cells infected with a vector with transcriptionally-competent (squares) or promotorless LTRs (triangles) as described in the text.

FIG. 14 shows the alteration in phenotype that occurred when cells were infected with a viral vector comprising a promoter insertion construct of the present invention. Individual clones of HCT9E cells that survived gancyclovir selection after infection with a transcriptionally-competent retroviral vector were expanded and equal number of cells plated in the presence or in the absence of puromycin. Puromycin resistance was compared to that of the parental cell line (HCT9E).

FIG. 15 is an example of transposon-based vector which can be used to introduce a promoter insertion construct of the present invention into a host cell. The structure of the construct in the genome prior to the first round of transposition. The transposon part is flanked by terminal fragments of Sleeping Beauty transposon (R). Hybrid puΔtk protein (fusion between puromycin resistance protein and a modified HSV-1 thymidine kinase) is expressed from a ubiquitous phosphoglycerate kinase promoter (PGK) and is supplemented with transcription termination and polyadenylation signals from simian virus 40 (pA). Human cytomegalovirus immediate early promoter is oriented towards the host DNA. Simian Virus 40 promoter and enhancer region (SV40) drives transcription towards the transposon. The gene for a fusion polypeptide (HygroLacZ), which consists of hygromycin resistance protein and E. coli β-galactosidase, is outside the transposon followed by additional polyadenylation and transcription termination signals. As shown, the HygroLacZ gene is silent due to the lack of a promoter.

### DETAILED DESCRIPTION OF THE INVENTION

In the description that follows, a number of terms are used extensively. The following definitions are provided to facilitate understanding of the invention.

The term “a” or “an” as used herein means one or more.

The term “expression” as used herein refers to the transcription of the DNA of interest, and, if applicable, the splicing, processing, stability, and, optionally, translation of the corresponding mRNA transcript.

“Gene” as used herein refers to any and all discrete coding regions of the cell's genome, as well as associated noncoding and regulatory regions.

“Mutagenized cell” as used herein refers to a host cell whose genome comprises a promoter insertion construct of the present invention.

“Mutant cell” as used herein refers to a mutagenized cell that exhibits a mutant phenotype, i.e., a phenotype that is different from the control phenotype of the non-mutagenized host cell.

“Control sequences” as used herein refers to components which are necessary or advantageous for the expression by a nucleic acid of a polypeptide or an RNA product, or both. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences typically include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably or operationally linked” as used herein refers to a configuration in which a control sequence is appropriately placed in the proper orientation and spacing relative to the coding sequence of a DNA fragment such that the control sequence directs the expression of a polypeptide or RNA product, or both encoded by the DNA fragment. The RNA product can be a sense or antisense molecule.

“Marker gene” as used herein refers to a nucleic acid encoding a product which directly or indirectly permits identification of cells comprising and expressing such gene. Marker genes as used herein encompass both screenable and selectable marker genes. As used herein, the term marker gene encompasses both a complete marker gene, i.e., a gene that includes control elements required for expression of the marker gene, as well as a coding sequence, and an incomplete marker gene that lacks one or more control sequences. One example of an incomplete marker gene is a promoterless marker gene.

“Promoter” as used herein refers to a nucleotide sequence that directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993)), cyclic AMP response elements (CREs), serum response elements (SREs; Treisman, Seminars in Cancer Biol. 1:47 (1990)), glucocorticoid response elements (GREs), and binding sites for other transcription factors, such as CRE/ATF (O'Reilly et al., J. Biol. Chem. 267:19938 (1992)), AP2 (Ye et al., J. Biol. Chem. 269:25728 (1994)), SPI, cAMP response element binding protein (CREB; Loeken, Gene Expr. 3:253 (1993)) and octamer factors (see, in general, Watson et al., eds., Molecular Biology of the Gene, 4th ed. (The Benjamin/Cummings Publishing Company, Inc. 1987), and Lemaigre and Rousseau, Biochem. J. 303:1 (1994)). If a promoter is an inducible promoter, then the rate of transcription increases in response to an inducing agent. In contrast, the rate of transcription is not regulated by an inducing agent if the promoter is a constitutive promoter. Repressible promoters are also known.

“Positive selectable marker gene”* as used herein refers to a nucleic acid whose product confers resistance of a cell to a predefined set of extracellular conditions. Most commonly such conditions involve exposure of the cells to specific cytotoxic or cytostatic compounds. Examples of positive selectable marker genes include antibiotic resistance genes, such as the neomycin resistance gene, the puromycin resistance gene, the hygromycin resistance gene, and the zeocin resistance gene; chemotherapeutic drug resistance genes, such as MDR-1 gene encoding P-glycoprotein; genes determining increased resistance to metabolic inhibitors, such as dihydrofolate reductase gene.

“Negative selectable marker gene”* as used herein refers to a nucleic acid whose product renders a cell sensitive to a specific condition. Most commonly such conditions involve exposure of the cells to specific cytotoxic or cytostatic compounds. Examples of a negative selectable marker gene include the Herpes Simplex Virus thymidine kinase (HSV-TK) gene and E. coli xanthine-guanine phosphoribosyltransferase (gpt) gene. Expression of the HSV thymidine kinase in a cell renders such cell sensitive to certain thymidine analogs, such as gancyclovir (GCV). Expression of the gpt gene renders cells sensitive to certain purine analogs, such as 6-thioguanine and 6-thioxanthine.

*Depending on the specific conditions and the genotype of the cell, certain marker genes could serve both as a positive selectable marker gene and a negative selectable marker gene. For example, xanthine-guanine phosphoribosyl transferase could make the cells that were otherwise depleted of such enzymatic activity sensitive to 6-thioxanthine, but resistant to HAT selection medium.

“Promoterless marker gene” as used herein refers to a modified marker gene that lacks a promoter element active in a given host cell.

“Splice acceptor site” as used herein refers to a sequence motif that specifies the 3′-terminus of an intron.

“Splice donor site” as used herein refers to a sequence motif that specifies the 5′-terminus of an intron. Splice donor and splice acceptor sites are paired if they in combination determine the boundaries of a removable intron.

“Unpaired splice donor site” is defined herein as a splice donor site present without a paired downstream splice acceptor site.”

The present invention provides methods and constructs for reliably, easily, and efficiently identifying genes whose products are involved in modulating select biological processes. The promoter insertion constructs of the present invention includes sequences that, under specific conditions, promote rearrangements which lead to the loss of a functional relationship between the promoter element and adjacent downstream host DNA. In a preferred embodiment the promoter element is flanked by recognition sites of a recombinase enzyme, so that introduction or activation of a site-specific recombinase into the mutagenized cell results in deletion of the promoter element from the mutagenized cell's genome. In another preferred embodiment, the promoter element is flanked by recognition sites configured such that introduction or activation of recombinase in the mutagenized cell results in inversion of the promoter element orientation with the mutagenized host cell's genome. In another preferred embodiment, the integrated promoter element is flanked by the recognition sites of a transposase protein, so that introduction or activation of the transposase in the mutagenized host cell would result in removal of the construct from its original integration site.

In yet another embodiment of the promoter insertion construct, a single recombinase recognition site is situated downstream of the promoter element, so that the operational linkage between the promoter element and the adjacent DNA of the mutagenized cell may be disrupted by inserting an additional DNA fragment via introduction of a plasmid, which harbors a single recombinase recognition site, into a mutagenized cell which contains the corresponding recombinase enzyme.

The promoter insertion construct may also include additional sequences (preferably ones encoding selectable marker genes) that facilitate identification or selection of cells that harbor the construct. In another embodiment the promoter insertion construct may include elements that facilitate selection of cells which harbor the vector integrated into a coding region of the host cell's genome. Sequences that allow detection of the recombination or transposition event could be present in the construct (for example, but not limited to positive or negative selection markers).

In another aspect, the invention provides a promoter insertion vector (for example, but not limited to a retrovirus- or transposon-based vector), which can be used to deliver the promoter insertion construct of the present invention into a host cell. The promoter insertion vector carries at least one promoter element engineered in such a way that, upon integration into the host genome, it will promote transcription of adjacent host DNA. In one embodiment a retrovirus-based vector is used to deliver the promoter element into the host cell via transduction. In another embodiment, a transposon-based vector is used to deliver the promoter element into a host cell comprising transposase. In another embodiment, a transposon-based vector is first stably integrated into the genome of the host cells, and subsequently mobilized from its location in the host cell's genome by temporary-limited activation of the transposase.

The present methods comprise introducing a promoter insertion construct of the present invention into the genomes of a collection of host cells having a predetermined, i.e., control, phenotype to provide a population of mutagenized cells; selecting mutagenized cells exhibiting a mutant phenotype of interest; treating the mutant cells with a disrupting agent that has recombinase activity; characterizing the phenotype of the treated cells; and correlating the phenotypes of the treated cell or the phenotypes of both the untreated and treated mutant cells with changes or lack thereof in the status of the linkage between the promoter element of the promoter insertion construct and adjacent host genomic DNA sequences that are downstream of the promoter element in the untreated mutant cells to identify genomic DNA coding sequences whose products are directly or indirectly involved in causing the mutant phenotype.

The methods of the present invention are an improvement over previous methods that involved inserting a DNA fragment into the genome of a cell and identifying the cell genomic sequences that are immediately upstream, i.e., that are linked to the 5′ end, or that are immediately downstream, i.e. that are linked to the 3′ end, of the inserted DNA fragment. These previous methods, which sometimes involve inserting a DNA fragment that comprises a promoter into the genome of the host cells, are hampered by the their inability to easily, quickly and cost-effectively demonstrate that the genomic sequences that are adjacent to and linked to the inserted fragment are the cause of the mutant phenotype. These previous methods are especially problematic in cases where a large number of spontaneous mutants arise in the mutagenized hosts cells independent of the random insertions. As a result of this large background of spontaneous mutants, previous methods have proven, in many instances, to be time-consuming, difficult, and in many instances, unproductive. The present method significantly reduces this background, and thus reduces the amount of time and cost required to identify the gene or genes that directly or indirectly cause the mutant phenotype, as well as increasing the likelihood that such genes will be found.

Previous methods that involve inserting a DNA comprising a promoter into the genome of a cell and identifying the cell genomic sequences that are linked to the 5′ end of the 3′ end of the inserted DNA fragment can also be problematic unless conditions are such that only a single copy of the DNA fragment is inserted into the genome of each host cell. If multiple copies of the previous DNA fragments are inserted into the cell, then the time, cost, and effort in determining which, if any of the inserts are linked to genomic sequences that cause the change in phenotype can be significant. The present method which employs promoter insertion constructs whose operational linkage with downstream genomic DNA coding sequence can be disrupted and then analyzed by various techniques overcome many of these problems.

In certain embodiments, the present methods employ a cell-based selective system and an insertional mutagenesis vector. The insertional mutagenesis vector is used to introduce at least one promoter element into non-targeted locations in the genome of the cells of the selective system. Such host cells are selected for the phenotype of interest, and the causative role of the inserted promoter element in altering the phenotype of the host cell is verified using a site-specific recombination-based validation procedure. A novel feature of the present method is the use of a promoter insertion construct that can be structurally disrupted or inactivated via a site-specific DNA rearrangement, thereby providing a sensitive test for the causative role of the promoter inserts in altering the host cell's phenotype.

The present methods may be carried out in any cell of eukaryotic origin, such as fungal, plant or animal. In preferred embodiments, the present methods are carried out in mammalian cells, including but not limited to rat, mouse, bovine, porcine, sheep, goat and human cells.

Additional features and advantages of the invention will be set forth in part in the description that follows, and in part will be obvious from the description, or may be learned by practice of the invention. The features and advantages of the invention will be realized and attained by means of the elements and combinations particularly pointed out in the appended claims.

Promoter Insertion Construct

The promoter insertion construct of the present invention is engineered to promote expression of a DNA sequence that is located in the genome of the host cell into which such promoter insertion construct is integrated. The promoter insertion construct comprises a transcriptional regulatory sequence, i.e., a promoter element and preferably an enhancer, for driving expression of host cell genomic sequences that are operably linked to the promoter insertion construct. The promoter insertion construct is also engineered such that the operable linkage between the inserted promoter element and the host cell genomic sequence can be disrupted, preferably by excision or rearrangement of the promoter element. Accordingly, in a highly preferred embodiment, the promoter element is flanked by an upstream recombinase recognition site sequence and a downstream recombinase recognition site sequence. The promoter insertion construct may also comprise a marker gene, preferably a selectable marker gene. Such selectable marker gene encodes a molecule which directly or indirectly allows for selection of host cells that comprise the promoter insertion construct. The promoter insertion construct of the present invention lacks or is free of a transcription terminator downstream of the promoter element. In certain embodiments, the promoter insertion construct of the present invention also lacks splice donor and splice acceptor sequences, while in other embodiments the promoter insertion construct comprises splice donor sequences to reduce or eliminate linkage of the construct to intron sequences than can potentially interfere with expression of the downstream genomic DNA sequences. The promoter insertion construct may also comprises sequences that can be used to identify a cell or DNA molecule that harbors the construct.

A. Promoter Element

The promoter insertion construct comprises a promoter. The promoter may be derived from the same species of organism as the host cell. Alternatively, the promoter may be derived from a different species or organism. The promoter may be an animal cell promoter, a plant cell promoter, a fungal cell promoter, or a viral cell promoter. The promoter may be a constitutive viral or cellular promoter, an inducible cellular promoter, or a tissue specific cellular promoter. Examples of suitable promoters include, but are not limited to the CMV immediate early gene promoter, an SV40 T antigen promoter, a β-actin promoter, the tetracycline regulated promoter (e.g. as described in Gossen, 1992), the herpes simplex thymidine kinase promoter, cytomegalovirus (CMV) promoter/enhancer, SV40 promoters, pga promoter, regulatable promoters (e.g., metallothionein promoter), adenovirus late promoter, vaccinia virus 7.5K promoter, and the like, as well as any permutations and variations thereof, which can be produced using well established molecular biology techniques (see generally, Sambrook et al. (1989) Molecular Cloning Vols. I-III, Cold Spring Harbor Laboratory Press, Cold Spring Harbor, N.Y., and Current Protocols in Molecular Biology (1989) John Wiley & Sons, all Vols. and periodic updates thereof, herein incorporated by reference).

Preferably, the promoter insertion construct also comprises an enhancer. The enhancer may be derived from the same organism and type of cell as the promoter or from a different organism or type of cell. Promoter/enhancer regions can also be selected to provide tissue-specific expression.

B. Recognition Sites for the Disrupting Agent

The present insertion construct also comprises a recognition site for a disrupting agent having site-specific recombinase activity. Site-specific recombinase activity is a biochemical property of a substance or of a mixture of substances to promote site-specific recombination. Site-specific recombination involves reaction between specific sites that are not necessarily homologous. During this reaction, breaks occur at or near the specific sites in the individual strands of two duplex DNA molecules or in two locations within the same duplex DNA molecule, or both, following by re-joining of the ends in a cross-wise manner. The individual steps of this process are not necessarily catalyzed by the same substance or a mixture of substances. This process requires recognition of specific sequence motifs and, hence, is distinct from the general homologous recombination process, which is driven by sequence homology of the substrate DNA molecules. The examples of substances with site-specific recombinase activities include, but are not limited to, integrase from phage lambda, E. coli resolvase XerD, Flp invertase from Saccharomyces cerevisiae, Cre recombinase from phage PI, etc. It is preferred that the site-specificity of a given disrupting agent is such that the specific sites rare in or are absent from the genome of the host cell prior to the introduction of the promoter insertion construct. In a highly preferred embodiment, the promoter insertion construct comprises two site-specific recombinase recognition site sequences that flank the promoter and, thus, under certain conditions, allow for rearrangement of the promoter element within its original integration site or excision of the promoter from its original integration site.

One example of a site-specific recombinase recognition site is a site that is recognized by a recombinase enzyme, e.g., the LoxP recombination sequence. Expression of cre recombinase in a cell whose genome comprises a promoter element flanked by two LoxP recombination sequences permits removal of the promoter element from its integration site. Other examples of suitable site-specific recombinase recognition site sequences are represented by terminal sequences of Sleeping Beauty transposon that could facilitate transposition of a fragment flanked by such sequences by the means of recruiting the Sleeping Beauty transposase (as described in Ivisc, 1997 and Vigdal, 2002”. (Ivics Z, Hackett P B, Plasterk R H, lzsvak Z. Molecular reconstruction of Sleeping Beauty, a Tc1-like transposon from fish, and its transposition in human cells. Cell. 1997 Nov. 14;91(4):501-10; Vigdal T J, Kaufman C D, Izsvak Z, Voytas D F, Ivics Z. Common physical properties of DNA affecting target site selection of sleeping beauty and other Tcl/mariner transposable elements. J. Mol. Biol. 2002 Oct. 25;323(3):441-52.).

In an alternative embodiment, the promoter insertion construct comprises a single recognition site for the disrupting agent. Such site is within or immediately downstream of the promoter element. In methods which employ such alternative embodiment, the host cells are co-transfected with a disrupter plasmid that carries another recombinase recognition site sequence for the same enzyme and a marker gene for identifying or selecting cells that have undergone a recombination event.

C. Marker Gene

In certain embodiments, a screenable marker gene or a selectable marker gene is included in the promoter insertion construct. Preferably, the marker gene encodes a full length-protein or transcript. The selectable marker gene is a gene that, upon integration of a promoter insertion construct containing the selectable marker gene into the genome of the host, allows selection of a cell containing and expressing the selectable marker gene. Examples of suitable selectable marker genes include, but are not limited to, a neomycin resistance gene, a hypoxanthine phosphoribosyl transferase gene, a puromycin resistance gene, a dihydrooratase gene, a glutamine synthetase gene, a dihydrofolate reductase gene, a multidrug resistance 1 gene, an aspartate transcarbamylase gene, a xanthine-guanine phosphoribosyl transferase gene, an adenosine deaminase gene, and a thymidine kinase gene. A screenable marker gene is a gene that allows sub-vital detection of a cell expressing such gene. Examples of screenable marker genes include, but are not limited to, genes that encode green fluorescent protein, beta-galactosidase, or cell surface proteins.

In other embodiments, the promoter insertion construct comprises a marker gene that encodes a product which, by itself, does not permit identification of the cell, but modulates the status of other genes that do. For example, a recombinase can be used as a marker gene because it can turn onexpression of an appropriately engineered resistance gene (this is used to make a temporary up-regulation of a marker result in a permanent phenotype). A small fragment of beta-galactosidase (alpha-fragment) may also used as a marker to achieve full beta-galactosidase activity when the rest of the enzyme (beta-fragment) is already expressed in the cells (this so-called “alpha complementation” allows to save space in a vector). A highly specific protease or another modifying enzyme could also be used as a marker if it processes a pre-expressed GFP variant to change its fluorescence level or localization; a small inhibitory RNA, which could be delivered as a complete expression cassette of only ˜100 bp in length, could serve as a marker if it suppresses the activity of a pre-introduced gene (e.g. by suppressing HSV TK it causes GCV resistance).

The marker gene may be located upstream of the promoter, either between the promoter element and the upstream recombinase recognition site sequence or upstream of the upstream recombinase recognition site sequence. In those cases where the marker gene is between the promoter element and the upstream recombinase recognition site, the marker gene is, preferably, a complete marker gene with transcriptional control and termination/polyadenylation elements. The sequence of such complete marker gene may be co-linear or inverted with respect to the sequence of the promoter.

Alternatively, the marker gene may be located downstream of the promoter and downstream of the recombinase recognition site sequence or between the promoter element and the downstream recombinase recognition site sequence. In this case, the marker gene is a promoterless marker gene which is operably linked to the promoter element and lacks a transcription termination sequence. To improve operable linkage of the promoter element with the downstream host DNA, it is highly desirable to include an internal ribosome entry site downstream of the selectable marker gene. It is also desirable to include an uncoupled donor splice site downstream of the internal ribosome entry site

D. Other Elements

Additional elements may also be included in the promoter insertion construct of the present invention to increase the yield of mutant cells in mutagenized populations. In one embodiment a translation start site followed by an open reading frame and an unpaired splice donor site are located downstream of the promoter element. In another embodiment, three separate constructs are created and used similarly to the one described above. These three variants differ from each other in that the splice donor site is placed in different translational “reading frames” with respect to the translation initiation site. The methods of the present invention may employ

i) a promoter insertion construct that lacks a translation initiation site; or

ii) three promoter insertion construct variants, each of which comprises a translation initiation site and a splice donor site, wherein the splice donor site is placed in three different translational reading frames with respect to the translation site in the three variants, or

iii) both i and ii.

Preferably, the three promoter insertion construct variants also comprise an independent ribosome entry site (IRES) that facilitates expression of open reading frames that are downstream of the promoter element. Elements that facilitate identification of the integration site may also be added to the construct. These include, but are not limited to, bacterial antibiotic resistance genes and plasmid replication origins. For example, in these cases, recovery of the sequences adjacent to the integrated construct may be done by digesting the DNA from the mutagenized cells with a restriction enzyme not cutting inside the construct, circularizing the pool of fragments by self-ligation, transfecting the ligation mixture into bacteria and selecting bacterial colonies that express the marker from the original construct. Such bacterial colonies will carry both the original construct and the flanking host sequences together in a form of a circular plasmid.

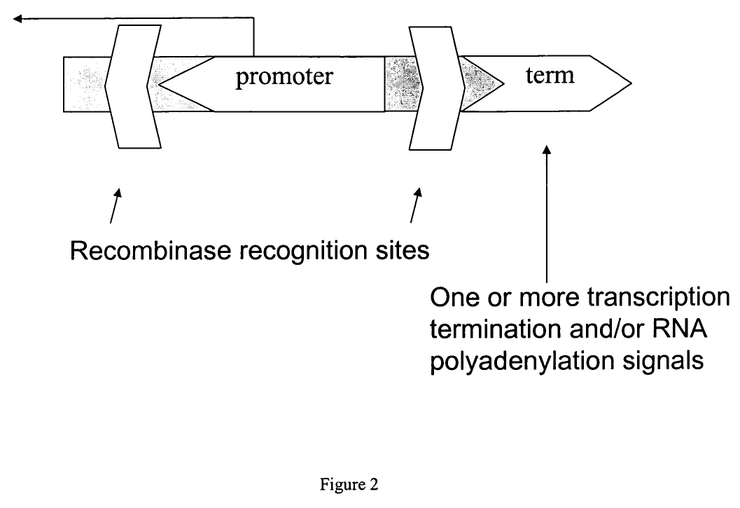

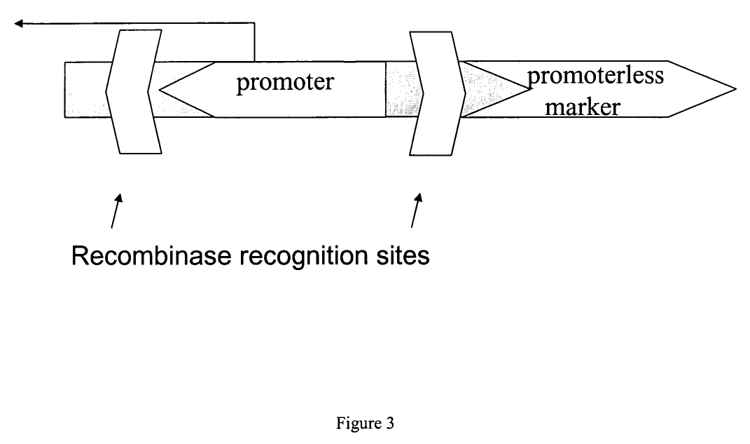

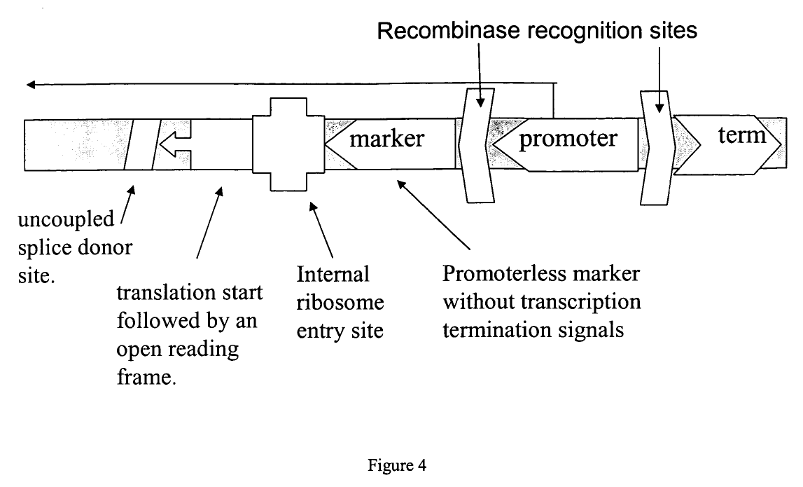

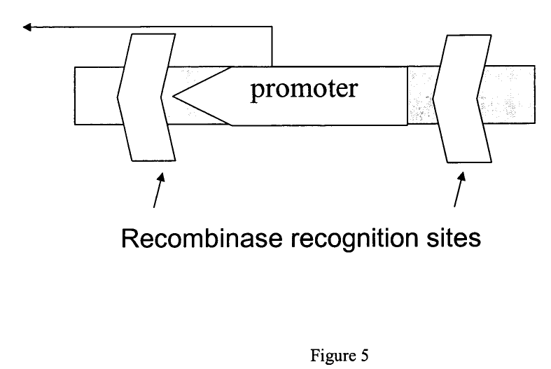

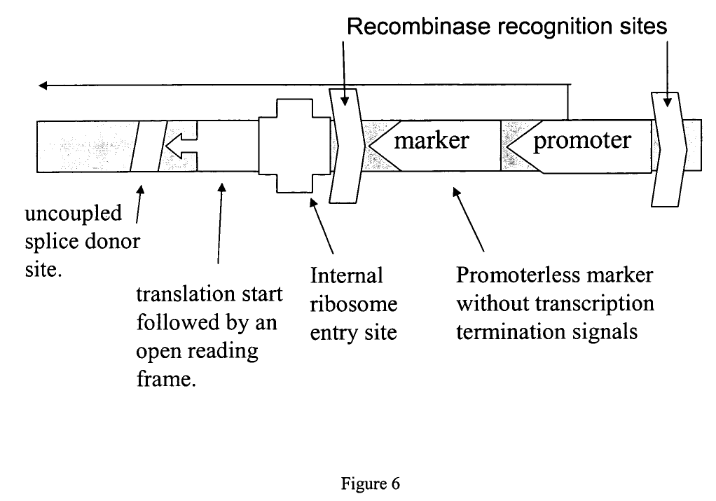

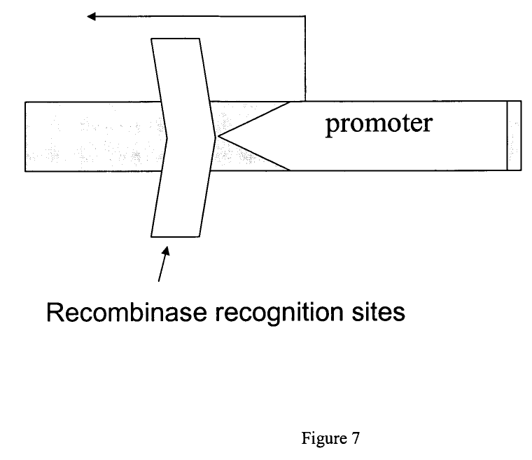

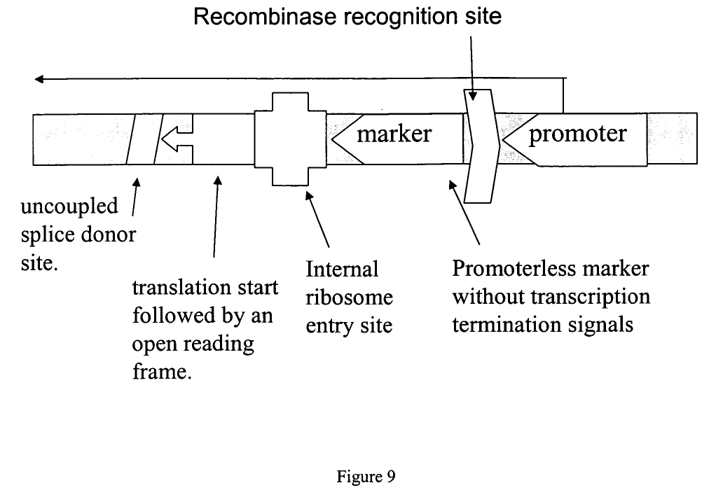

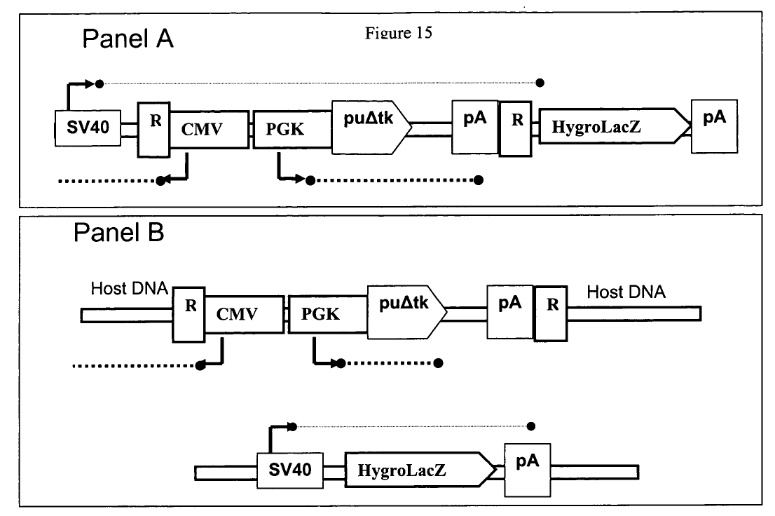

The promoter insertion construct of the present invention lack a transcription termination sequence downstream of the promoter element. The promoter insertion constructs of the present method are made using procedures known in the art. Non-limiting examples of the promoter insertion constructs of the present invention are shown in FIGS. 2-10 and 15 and described in Examples 1-3 below.

Introducing the Promoter Insertion Construct Into the Host Cells

Vectors incorporating the present promoter insertion constructs can be used to incorporate the present promoter insertion construct into the genome of virtually any type of eukaryotic cell. For example, vectors that incorporate the present promoter insertion constructs can be used to insert the construct into the genome of primary animal tissues as well as any other eukaryotic cell or organism including, but not limited to, yeast, molds, fingi, and plants. Additional examples of suitable target cells include, but are not limited to, mammalian, including human, endothelial cells, epithelial cells, islets, neurons or neural tissue, mesothelial cells, osteocytes, lymphocytes, chondrocytes, hematopoietic cells, immune cells, cells of the major glands or organs (e.g., lung, heart, stomach, pancreas, kidney, skin, etc.), exocrine and/or endocrine cells, embryonic and other stem cells, fibroblasts, and culture adapted and/or transformed versions of the above can be used in conjunction with the described vectors. Additionally, tumorigenic or other cell lines can be targeted by the presently described vectors

Vectors comprising the present constructs can be introduced into target cells by any of a wide variety of methods known in the art. Examples of such methods include, but are not limited to, electroporation, viral infection, retrotransposition, microinjection, lipofection, or transfection.

Vectors comprising the present constructs can also be used in virtually any type of phenotypic or genetic screening protocols both in vitro and in vivo, and the presently described vectors provide the additional advantage of enabling rapid methods of identifying the DNA sequences of the genes that are operably linked to the promoter elements of the present constructs and confirming that such genes are the direct or indirect cause of the altered phenotype.

Suitable vectors that can be used in conjunction with the presently disclosed promoter insertion construct include, but are not limited retroviral vectors, lentiviral vectors, transposon-based vectors, and T-DNA based vectors.

In certain embodiments the vector delivering the promoter insertion is a retroviral vector. The vector is delivered via retroviral infection by the techniques widely known in the art. The retroviral vectors are preferred for a broad range of susceptible cells, which could be infected relatively easily and efficiently. In the preferred embodiment such a vector contains retroviral long terminal repeats (LTRs) that are inactivated upon integration (“self-inactivating vector”), a set of sequences essential for packaging into viral particle and integration in a host cell genome, and the promoter used for insertional mutagenesis. In the most preferred embodiment such promoter is a regulated promoter (for example, tetracycline-regulated promoter) and is positioned opposite to the retroviral LTRs. This design ensures that the promoter-driven transcript is not terminated at the natural termination and polyadenylation sites within the LTRs, and that the promoter is silent during production of the virus to minimize interference with transcription of the entire construct. In turn, LTR promoter in a self-inactivating vector, inactivated after integration, should not interfere with the regulated promoter function. Methods which employ a retroviral-based vector are described in Examples 1 and 2 below.

In other preferred embodiments, the vector delivering the promoter insertion construct is a transposon-based vector. In these embodiments at least one promoter is situated close to and oriented towards the transposon termini. Transposon vectors may be preferred because their structure is not limited by the requirements of packaging. They also may be preferred because the length of the minimal sequences sufficient for transposition is shorter than that of the minimal sequences required for delivery of a retroviral vector. Transposons that transpose through a conservative mechanism (that is, a transposon predominantly moves from one location to the other, rather then creating a new copy at the new location) may be preferred because the entire construct may be removed with minimal remaining “footprint” (as small as two base pairs). Finally, transposon-based vectors may be preferred because they could act in a cell autonomous manner, that is, a transposon may be pre-integrated in a cell and then mobilized by temporally-limited expression of transposase. The vector may also contain additional elements for example, positive or negative selection markers. An example of a method which employs a transposon-based vector of the present invention is described in Example 3 below.

Other examples of the delivery system may include delivery of the construct in the form of DNA via transfection by the techniques commonly known in the art, in the form of agrobacterium T-DNA (for delivery into plant cells), or another random inserting viral-based vector. Such delivery systems are known in the art. Transfection with plasmid DNA is less preferred because it generally does not preserve the whole structure of the construct and produces greatly variable number of inserts.

Characterization of the Altered Phenotype in Mutant Cells

The promoter insertion construct is introduced into a collection of cells having a predetermined or control phenotype. Integration of construct into the genome of such cells changes the function of one or more of the host cell genes resulting in a detectably altered phenotype which allows for identification of cells harboring such changes. In the preferred embodiment, reversion of such a change also results in reversion to the predetermined phenotype.

A. Host Cells That Comprise a Genetically-Engineered Selection System

In one preferred embodiment the host cells comprise a genetically-engineered selection system. Such selection system comprises a known promoter, i.e., the promoter of a known gene or a promoter that is known to be responsive to certain transcription factors, such as p53 or NFkB. The known promoter is operably linked to one or more marker genes. In the preferred embodiments, the marker genes are positive selectable marker genes or negative selectable marker genes, or both. In some embodiments, the control or predetermined phenotype of cells comprising the genetically-engineered selection system is specific resistance provided by expression of the positive selectable marker gene and specific sensitivity due to the expression of the negative selectable marker gene. In other embodiments, the control phenotype is failure to express the positive selectable marker gene or the negative selectable marker gene or both. Cells which exhibit a reversal of the resistance pattern, i.e., an altered phenotype, following integration of the promoter insertion construct of the present invention into the cells' genome are used to identify endogenous genes that are operably linked to the promoter of the present promoter insertion construct and whose products are transcription factors or factors involved in modulating the function of the transcription factors that upregulate the known promoter or that act as co-factors of such transcription factors. Preferably, the selection system construct is integrated into the genome of the cell or present as an episome.

B. Host Cells That do not Comprise a Genetically-Engineered Selection System

In alternative embodiments, the cells do not comprise a genetically-engineered selection system. In such cells the changes in endogenous gene expression due to integration of the promoter insertion construct into the host cell's genome may result in increased tolerance to specific conditions including, but not limited to, tolerance to otherwise toxic chemical agents or to the lack of otherwise essential components of growth medium. In this case, the mutant cells may be isolated by subjecting a mixed cell population of the mutant cells to such specific culture conditions. Examples of such phenotypic changes include, but are not limited to, elevated tolerance to antibiotics, chemotherapeutic agents, growth factor withdrawal and nutrient withdrawal.

In another embodiment, integration of the promoter insertion construct and changes in expression of host cell genes that are operably linked to the promoter of the construct may result in the altered expression or activity of cellular factors that permits sub-vital identification and isolation of a cell harboring such alterations. Examples of such factors include, but are not limited to, cell surface markers (allow for affinity separation or for affinity labeling followed by separation according to the properties of the label, such as fluorescence or magnetism), enzymes (allow for a chemical reaction, products of which mark the cell for isolation) or fluorescent proteins.

In another embodiment, integration of the present promoter insertion construct into the host cell's genome and changes in expression of host cell genes that are operably linked to the promoter of the construct may produce a phenotypic alteration which includes a visually identifiable morphological change that allows for identification of an individual cell or a cell colony which harbors such changes. Examples of such a change include, but are not limited to, a morphological transformation that is characterized by formation of foci in confluent cultures or a failure to display the features of differentiation, such as accumulation of fat deposits in adipocytes.

In another embodiment, the cells of the selective system are the cells of a living organism and the phenotypic change includes alterations in traits that are detectable at an organismal level, such as formation of tumors or other morphologically distinct structures.

In all embodiments the cells that compose the selective system may have been genetically engineered for the expression or for the lack of certain factors to permit the use of detection methods mentioned above. The cells with desirably altered phenotypes are identified and selected for further work.

The animals and cells produced using the presently described vectors are useful for the study of basic biological processes and diseases including, but not limited to, aging, cancer, autoimmune disease, immune disorders, alopecia, glandular disorders, inflammatory disorders, ataxia telangiectasia, diabetes, arthritis, high blood pressure, atherosclerosis, cardiovascular disease, pulmonary disease, degenerative diseases of the neural or skeletal systems, Alzheimer's disease, Parkinson's disease, asthma, developmental disorders or abnormalities, infertility, epithelial ulcerations, and viral and microbial pathogenesis and infectious disease (a relatively comprehensive review of such pathogens is provided, inter alia, in Mandell et al., 1990, “Principles and Practice of Infectious Disease” 3rd. ed., Churchill Livingstone Inc., New York, N.Y. 10036, herein incorporated by reference). In addition to the study of diseases, the presently described cells, and animals are equally well suited for identifying the molecular basis for genetically determined advantages such as prolonged life-span, low cholesterol, low blood pressure, resistance to cancer, low incidence of diabetes, lack of obesity, or the attenuation of, or the prevention of, all inflammatory disorders, including, but not limited to coronary artery disease, multiple sclerosis, rheumatoid arthritis, systemic lupus erythematosus, and inflammatory bowl disease.

Validation of the Integration Event as the Causative Factor of the Altered Phenotype

The present method also includes validation of individual integration events as the causative factors in the altered phenotypes of the selected mutants. This is initiated by disrupting the operable linkage between the promoter insertion construct and the adjacent genomic DNA via site-specific recombination using the sequences pre-engineered into the promoter insertion construct. The relevant recombinase (for example, a transposase or a recombinase enzyme) may be introduced into the cells as a protein or as an expression construct after the promoter insertion construct is introduced into the host cell. The enzyme or expression construct could also be introduced into the host cell prior to introduction of the promoter insertion construct into the host cell, and either production or activation of the enzyme would be induced at the validation step. Introduction, regulated expression and controlled activity of such an enzyme have been widely reported in the art. If more then a single integration per cell is anticipated, recombination, preferably, is performed in conditions where it is less than 100% efficient.

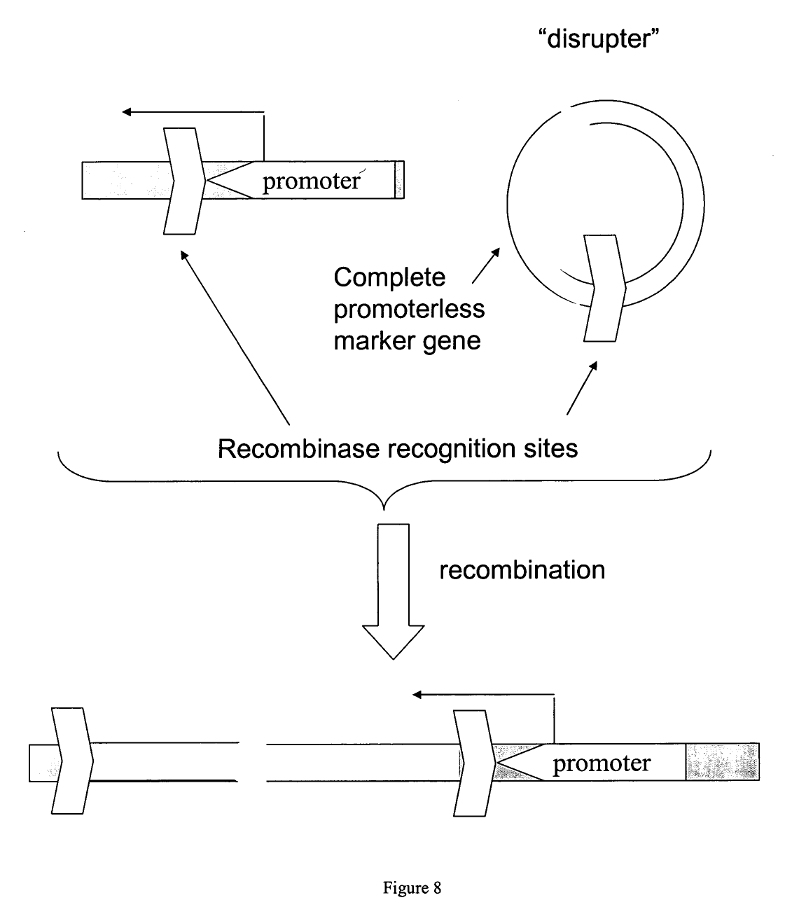

In certain embodiments, a separate “disrupter” plasmid is introduced into the mutant cells together with the recombinase enzyme to be integrated downstream of the insertional promoter element in order to disrupt the operable linkage between the insertional promoter element and the host DNA. (See FIG. 8.)

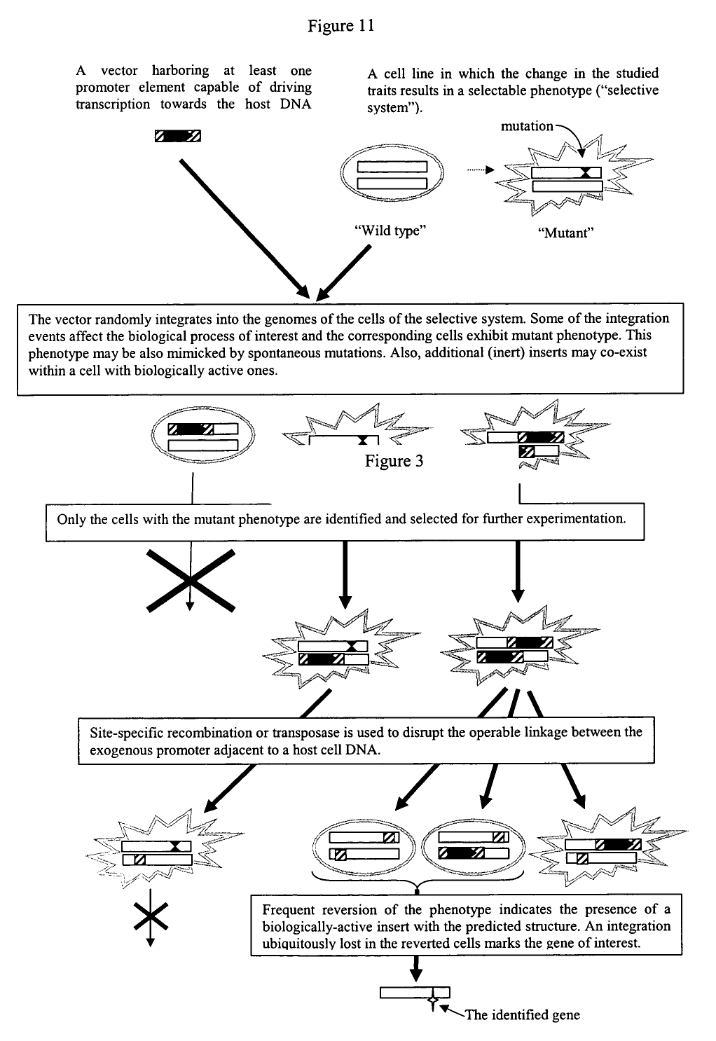

In a preferred embodiment, the incidence of mutagenized mutant cells that revert to the predetermined or control phenotype upon the presumed inactivation of the operable linkage between the inserted promoter element and the mutagenized host cell's endogenous gene is scored in a progeny of an individual mutant and compared to that of spontaneous reversion rate among the same cells. (See FIG. 11) Increase in reversion rate upon site-specific recombination indicates that the altered phenotype is the consequence of the promoter insertion and that an endogenous gene at the site of integration is involved in the biological process of interest. If more than one integration site is present, it is expected that in the revertants, the promoter construct could still be found at the inert sites, but essentially never at the site primarily responsible for the phenotype of interest. Thus, the comparison of the insert pool after and prior to recombination/selection step allows identification of the position of the relevant endogenous genes.

Characterizing the Operational Linkage Between the Promoter and Downstream Host DNA in Treated and Untreated Mutant Cells

The method also comprises investigating the operational linkages between the promoter element and the adjacent downstream genomic DNA sequences in mutant cells that have and have not been treated with the disrupting agent and then correlating such operational linkages with the phenotypes of such cells. Standard molecular methods, such as sequencing, can be used in this investigation. In a preferred embodiment the operational linkage is examined via “inverse PCR” (“iPCR”) (Ochman, 1988). Examples of methods which employ iPCR include, but are not limited to, the following:

1. Clone multiple “inverse” PCR products from each sample. Individually sequence the cloned fragments.

2. Resolve “inverse” PCR products from the treated and untreated mutant cells by gel electrophoresis. Compare the patterns of the bands. Excise from the gel and clone the fragment seen only in the untreated cells.

3. Clone the “inverse” PCR fragments from untreated cells in a vector that supports easy identification of insert-bearing plasmids (e.g. pCR2.1 from Invitrogen, which allows to distinguish native and insert-bearing plasmids by the color of colonies on the appropriate growth medium). Make replicas of the plates and transfer colonies to a membrane for hybridization as described (Sambrook, J., Fritsch, E. F., Maniatis, T. “Molecular Cloning: A laboratory manual,” Cold Spring Harbor Laboratory Press, 1989). Use the pooled PCR products from puromycin-resistant cells as a probe for hybridization with the membrane. Caution has to be taken to avoid cross-hybridization of different fragments via common sequences (e.g. primer-annealing regions). Appropriate primer design and high stringency of hybridization should resolve any artifacts of this sort. Under optimal conditions, the colonies that bear inserts, but fail to hybridize, are likely to carry differentially represented fragments.

### EXAMPLES

The following examples are for purposes of illustration only and are not intended to limit the scope of the invention as defined in the claims which are appended hereto. The references cited in this document are specifically incorporated herein by reference.

### Example 1

### A Genetic Screen for the Regulators and Co-Factors of p53

P53 is a transcription factor that is activated by a variety of stress stimuli, including DNA damage and activated oncogenes. Activation of p53 results in growth arrest or cell death and serves to prevent tumorigenesis and mutagenesis. P53 is arguably the quintessential tumor suppressor, which is frequently inactivated in human malignancies. Nevertheless, a substantial percentage of tumors maintain structurally unaltered p53, while somehow avoiding its growth suppressive activities. At least in some of such cases, elevation of p53 activity was shown to be suppressive for the cell growth, suggesting functional re-activation of p53 as a possible therapeutic strategy. Overall, positive regulators or co-factors of p53 are candidates for therapeutic activation, while negative regulators are possible targets for inhibition in human malignancies. Information about factors that are negative or positive regulators or co-factors of p53 is useful for the diagnosis and therapy of cancer. Hence, identification of such factors is an important task of biomedical science. Steps of one method for screening for or identifying regulators or co-factors of p53 are as follows:

1. Establish a selective system in which changes in p53 activity are associated with a selectable phenotype. Such a system is represented by a derivative of HT1080 fibrosarcoma cell line, which we designated HCT9. These cells express cDNA for puromycin resistance and thymidine kinase under the control of an artificial p53-responsive promoter as described earlier (Agarwal, 2000). Due to a relatively high activity of p53 in these cells, HCT9 are constitutively resistant to puromycin and sensitive to gancyclovir. The loss of p53 function is associated with reversal of the resistance pattern. Spontaneous reversal of the resistance pattern occurs at a frequency of

2. Develop a variant of the selective system highly susceptible to retroviral infection. This is an optional, but helpful step. We achieve this by ectopically expressing murine ecotropic receptor (Albritton, 1989). This makes human cells susceptible to infection with retroviral vectors typed with ecotropic envelope. Ecotropically typed particles are not infectious to humans and are easily obtained at high titers.

3. Develop a variant of the cell line from step 2 that supports tetracycline-regulated expression. This step is optional, but beneficial. It is achieved via ectopic expression of a hybrid protein (“tetracycline activator” or “TA”) that combines transcription activating domain of virion protein 16 of herpes simplex virus with a bacterial DNA-binding polypeptide of tetracycline repressor protein (Gossen, 1992).

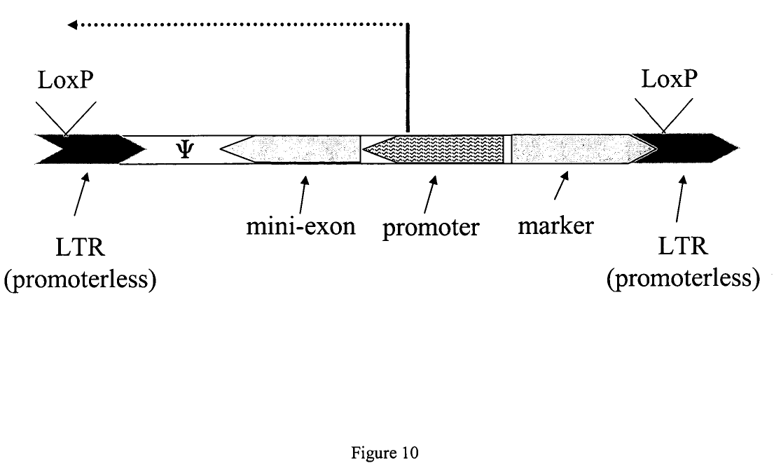

4. Construct a promoter insertion construct vector of the following structure using common molecular biology techniques (see FIG. 10). The vector (“promoter insertion vector”) preferably is constructed in 4 variants. Three of the variants differ from the one shown in the figure in that they have a translation initiation site and a splice donor site (“variable element”) inserted downstream of the regulated promoter. These latter three variants differ from each other in that the splice donor site would be placed in different reading frame with respect to the translation initiation site. Introduction of the translation start site and a splice donor is expected to facilitate production of truncated gene products when integration occurs in an intron collinearly with the gene. Since a priori one may not predict the reading frame of the target gene, for maximally comprehensive screening, it is desirable to use all three variants in the same experiment.

5. Deliver the promoter insertion vector into the target cells. Infectious particles are generated as described earlier (Pear, 1993). Deliver four variants of the vector on separate plates. Use multiple rounds of infection to achieve high infection efficiency.

6. Plate subconfluent (10-20% confluent) cultures of the infected cells and subject them to gancyclovir selection (2 microg/ml) until visible cell death has ceased and well-defined colonies are formed (7-10 days).

7. Pick individual colonies and expand them separately. Progeny of each colony are treated as a separate clone. Use an aliquot from each clone for cryopreservation and use the rest for testing. Expansion of the clones is done in the presence of antibiotic G418 (to eliminate the clones that appeared spontaneously and do not carry a vector insert) and tetracycline (to minimize the potentially detrimental effects of the regulated promoter). Subsequent experiments are performed in the absence of G418 and tetracycline, unless stated otherwise.

8. For each clone, separately transfect cells with a plasmid that expresses Cre recombinase or a control plasmid that does not express this enzyme. Transient transfection is achieved via methods commonly known in the art (e.g. via lipofection with Lipofectamine reagent from Invitrogen Corporation) Test the cells transfected with either plasmid for the ability to survive in the presence of puromycin (1 microg/ml). If expression of Cre has elevated the frequency of puromycin resistant colonies, the clone is taken for future analysis.

9. The Cre-transfected cells that survive in puromycin are pooled and their DNA extracted. Also, DNA is obtained from the cells of the same original clone that have not been exposed to Cre (“untreated cells”). Both DNA samples are used for Southern blotting to establish the number of distinct integration sites. For this experiment, the DNA samples are digested with an enzyme that cuts at least once in the targeting vector. A sequence that is expected to anneal to a variable-length fragment formed at the junction of vector and host DNA is chosen as the probe. Occasionally, two or more of such junction fragments may have lengths similar enough not to be resolved on the gel. In this case different restriction enzymes or enzyme combinations are employed until the total number of individual inserts is reliably quantified. Untreated cells should contain 1 or, less likely, 2 inserts that are missing in revertants. The likelihood of more than 2 inserts in a given cell contributing to the phenotype is negligible and loss of more than 2 inserts would indicate unacceptably high level of Cre activity at the transfection step. In this case, lower levels of Cre should be expressed (e.g. by adding less Cre-expressing plasmid to the transfection mixture or by expressing Cre from a less potent or regulatable promoter).

10. If a given clone contains only one viral insert and it is missing in Cre-induced revertants, perform “inverse” PCR* to recover the junction between the insert and the genomic DNA. Clone and sequence the PCR product. Identify position of the insert in the genome using appropriate database (e.g. human genome sequence provided by NCBI). The product of the gene at the integration site is considered a putative regulator or co-factor of p53. Proceed with further characterization using tests of p53 functions commonly known in the field.

11. If a given clone carries multiple inserts, perform “inverse” PCR* on both the untreated and puromycin-selected cells. Clone PCR products from the untreated cells in pCR2.1 cloning vector from Invitrogen (or any other vector that supports cloning of PCR products and allows for easy identification of colonies that carry inserts). It is highly desirable that the total number of inserts identified via “inverse” PCR matches that of the distinct junction fragments identified by Southern blotting. Since the length of the junction fragment could not be readily predicted and very long fragments are poorly amplifiable by PCR, one may try several different restriction enzymes or restriction enzyme combinations for the initial step of the “inverse” PCR procedure. Identify the PCR products that were obtained from untreated, but not from the puromycin-selected cells**. Sequence such product(s) and find location of the targeted gene, as in step 10. Proceed with studies of putative regulators or co-factors of p53 as in 10.

Inverse PCR for identifying an insert integration site was originally described in Ochman and Hart, 1988).

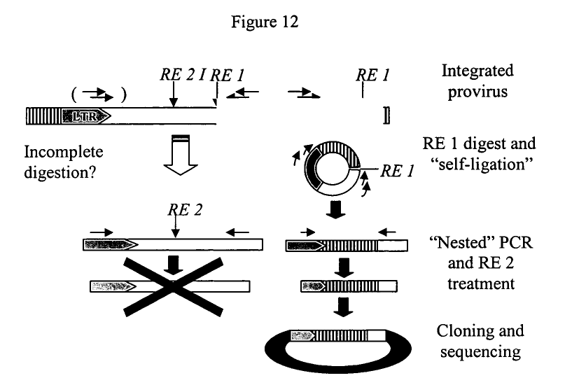

A schematic of the inverse PCR procedure is shown in FIG. 12. Thin horizontal arrows represent primer-annealing sites. Hatched bars represent the host genomic DNA. “LTR” refers to the retrovirus long terminal repeats modified as described above (e.g. bearing deletion and recombinase recognition sequences). “RE 1”—restriction enzyme that cuts at least once in the vector. “RE 2”—rare cutting restriction enzyme distinct from RE 1 that cuts at least once in the vector. Use of RE 2 is optional and is intended to remove possible contamination coming from incomplete digestion of genomic DNA (due to the repetitive nature of proviral termini, undigested provirus may serve as a template for PCR with the indicated primers.). RE 1 should always produce the same “overhangs” at the DNA termini to simplify the ligation step. It is highly desirable that RE 1 sites are present in the genomic DNA much more frequently than the sites for RE 2, so that the junction fragments had identical “RE 1″-type overhangs. Nested PCR is optional and helps to eliminate artifacts due to non-specific primer annealing within genomic DNA. As an additional safeguard against artifacts, correctly amplifies fragments should have sequence homology to the original vector extending beyond the primer sequence, as well as an RE 1 recognition site.

There are several approaches to identify the inserts differentially represented in two DNA samples. Here the examples of such approaches:

A. Clone multiple “inverse” PCR products from each sample. Individually sequence the cloned fragments. The number of distinct products should match the one predicted from Southern experiment. The inserts found in untreated, but missing in Cre-treated cells are taken for further work.

B. Resolve “inverse” PCR products from both samples by gel electrophoresis. Compare the patterns of the bands. The number of discrete bands should match the number of discrete junction fragments identified by Southern experiment. Excise from the gel and clone the fragment seen only in the untreated cells.

C. Clone the “inverse” PCR fragments from untreated cells in a vector that supports easy identification of insert-bearing plasmids (e.g. pCR2.1 from Invitrogen, which allows to distinguish native and insert-bearing plasmids by the color of colonies on the appropriate growth medium). Make replicas of the plates and transfer colonies to a membrane for hybridization as described (Maniatis). Use the pooled PCR products from puromycin-resistant cells as a probe for hybridization with the membrane. Caution has to be taken to avoid cross-hybridization of different fragments via common sequences (e.g. primer-annealing regions). Appropriate primer design and high stringency of hybridization should resolve any artifacts of this sort. Under optimal conditions, the colonies that bear inserts, but fail to hybridize, are likely to carry differentially represented fragments. Use these fragments for further studies.

### REFERENCES

Agarwal M L, Ramana C V, Hamilton M, Taylor W R, DePrimo S E, Bean L J, Agarwal A,

Agarwal M K, Wolfman A, Stark G R Regulation of p53 expression by the RAS-MAP kinase pathway. Oncogene 2001 May 3;20(20):2527-36

Albritton L M, Tseng L, Scadden D, Cunningham J M. A putative murine ecotropic retrovirus receptor gene encodes a multiple membrane-spanning protein and confers susceptibility to virus infection. Cell. 1989 May 19;57(4):659-66.

Anastassiadis K, Kim J, Daigle N, Sprengel R, Scholer H R, Stewart A F. A predictable ligand regulated expression strategy for stably integrated transgenes in mammalian cells in culture. Gene. 2002 Oct. 2;298(2):159-72.

Gossen M, Bujard H. Tight control of gene expression in mammalian cells by tetracycline-responsive promoters. Proc Natl Acad Sci USA. 1992 Jun. 15;89(12):5547-51.

Ochman H, Gerber A S, Hartl D L. Genetic applications of an inverse polymerase chain reaction. Genetics. 1988 November; 120(3):621-3.

Pear W S, Nolan G P, Scott M L, Baltimore D. Production of high-titer helper-free retroviruses by transient transfection. Proc Natl Acad Sci USA. 1993 Sep. 15;90(18):8392-6.

Additional information:

The effect of a functional promoter on the yield of phenotypic mutants in HCT9E cells.

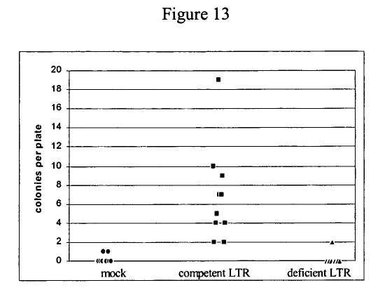

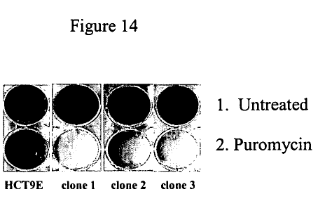

We have tested the feasibility of promoter insertion mutagenesis in the HCT9E system. We infected HCT9E cells with the pBNsLoxPGFP retrovirus, which carries promoter-competent LTRs (FIG. 13), or the pBNdLLoxP4 retrovirus with self-inactivating LTRs (similar to pBNdLLoxPGFP, but lacking LTR promoter function and GFP expression), or exposed cells to supernatant medium from packaging cells lacking infectious particles. Each was seeded on ten 15-cm plates, ˜55×10⁵ cells per plate, and subjected to GCV selection. While both constructs produced similar virus titers, only the one with the promoter-competent LTRs was able to increase the yield of GCV-resistant clones (FIG. 13). There was a substantial increase in the colony yield (7 vs. 0.2 colonies per plate; 10 vs. 1 positive plates) when a functional promoter was present. A sample of clones established from GCV-resistant colonies demonstrated loss of puromycin resistance (FIG. 14), indicating that the selection procedure indeed yields the cells with the expected phenotype. The experiment confirms that random promoter insertion is an efficient strategy to generate mutants in cultures of mammalian cells.

### EXAMPLE 2

### A Genetic Screen for Components of NFkB Signaling Pathway

NFkB is a family of proteins that act as transcription factors. Proteins of this family are activated in response to variety of stimuli, including cytokines, growth factors, stress, stimuli, etc. Activated NFkB, in turn, turns on expression of various target genes, including the ones involved in inflammatory response, cell survival and cell growth. NFkB appears as suitable therapeutic target in chronic inflammatory disease and cancer. It is desirable to identify the proteins that regulate NFkB activity since they may represent both markers and therapeutic targets for various diseases. Both positive and negative regulators of NFkB signaling pathway are expected to exist.

1. Establish a suitable selective system. A suitable selective system for NFkB-based genetic selection is 293 ZeoTK cell line (Li, 1999). These cells express thymidine kinase and zeocin resistance protein under the control of an NFkB—dependent promoter. While both markers are not expressed at significant levels during normal culture conditions, additional stimulation of NFkB (e.g. upon cytokine treatment) leads to accumulation of both proteins. Alternatively, thymidine kinase and zeocin resistance protein may accumulate if the pathway is turned on via activation of a positive regulator or inactivation of a negative regulator. In this case, a cell becomes constitutively resistant to zeocin and sensitive to gancyclovir (“suicide” substrate for thymidine kinase). Reversion of such a mutation is identifiable by re-gained resistance to gancyclovir in the absence of additional cytokine treatment.

2. Express murine ecotropic receptor in 293ZeoTK cells. The expression cassette for this protein is delivered via conventional techniques (e.g. DNA transfection). Expression of murine ecotropic receptor permits transduction with safe and efficient ecotropically typed retroviral vectors (Albritton, 1989), which otherwise infect only murine cells. Although this step is optional (retroviral vectors may be typed to infect human cells directly and non-retroviral delivery systems are available), we prefer this route due to the combination of safety and efficiency. The cell clone engineered this way and showing susceptibility to infection with ecotropic infection is designated “293ZeoTK/Eco”.

3. Express the “tetracycline activator” protein (TA) in 293ZeoTK/Eco. This is a chimerical protein containing parts from bacterial tetracycline repressor supplemented with a mammalian transactivation domain (Gossen, 1992). It binds to and promotes transcription from artificial promoters that combine minimal mammalian promoter with several tetracycline operator repeats. Upon addition of tetracycline, DNA binding is disrupted and transcription declines. Select a cell clone that supports tetracycline-sensitive expression. This clone is used to conduct the selection experiment. Use of a tetracycline-regulated expression is optional, but beneficial. It is desirable to limit the effects of the integrated promoter solely to the times when cells are being screened or selected for specific phenotypes, since prolonged activity of such a promoter in some cases may be detrimental to cell growth or cause additional (e.g. compensatory) changes.

4. Construct a vector of the following structure (referred to as “targeting vector” afterwards) using conventional molecular cloning techniques. The vector (“targeting vector”) should be constructed in 4 variants. Three variants should differ from the one shown in the figure in that they would have a translation initiation site and a splice donor site (“variable element”) inserted downstream of the regulated promoter. These latter variants would differ from each other in that the splice donor site would be placed in different reading frame in respect to the said translation initiation site. Introduction of the translation start site and a splice donor should facilitate production of truncated gene products when integration occurs in an intron collinearly with the gene. Since a priori one may not predict the reading frame of the target gene, for maximally comprehensive screening all three variants have to be used in the same experiment.

5. Deliver the targeting vector into the target cells. Infectious particles are generated as described earlier (Pear, 1993). Deliver four variants of the vector on separate plates. Use multiple rounds of infection to achieve high infection efficiency.

6. Place subconfluent (10-20%) cultures of infected cells in 50 microg/ml of zeocin and continue selection with periodic change of selective medium for 10-14 days, until visible cell death have ceased and well-defined colonies are formed. Spontaneous survival of the original 293ZeoTK cells in these conditions is between 1 out of 10⁵ and 1 out of 10⁶ cells.

7. Pick individual colonies and expand them separately. Progeny of each colony is treated as a separate clone. Use an aliquot from each clone for cryopreservation and use the rest for testing. Expansion of the clones is done in the presence of tetracycline (to minimize the potentially detrimental effects of the regulated promoter). Subsequent experiments are performed in the absence of tetracycline, unless stated otherwise.

8. In two separate wells transfect cells from each clone with a Cre-expressing plasmid or with a control plasmid that does not express Cre. Transient transfection is achieved via methods commonly known in the art (e.g. via lipofection with Lipofectamine reagent from Invitrogen Corporation). Estimate the yield of gancyclovir-resistant cells among cells transfected with a Cre-expressing or control plasmids. The clones that show increase in the yield of gancyclovir-resistant cells upon transfection with a Cre-expressing plasmid are used for further studies.

9. The Cre-transfected cells that survived in gancyclovir are pooled and their DNA extracted. DNA is also obtained from the cells of the same original clone that have not been exposed to Cre (“untreated cells”). Both DNA samples are used for Southern blotting to establish the number of distinct integration sites. For this experiment, the DNA samples are digested with an enzyme that cuts at least once in the targeting vector. As a probe one should choose a sequence that is expected to anneal to a variable-length fragment formed at the junction of vector and host DNA. Occasionally, two or more of such junction fragments may have lengths similar enough not to be resolved on the gel. In this case one should try different restriction enzymes or enzyme combinations until the total number of individual inserts is reliably quantified. Untreated cells should contain 1 or, less likely, 2 inserts that are missing in revertants. The likelihood of more than 2 inserts in a given cell contributing to the phenotype is negligible and loss of more than 2 inserts would indicate unacceptably high level of Cre activity at the transfection step. In this case, lower levels of Cre have to be expressed (e.g. by adding less Cre-expressing plasmid to the transfection mixture or by expressing Cre from a less potent or regulatable promoter).

10. If a given clone contains only one viral insert and it is missing in Cre-induced revertants perform “inverse” PCR* to recover the junction between the insert and the genomic DNA. Clone and sequence the PCR product. Identify position of the insert in the genome using appropriate database (e.g. human genome sequence provided by NCBI). The gene at the integration site is considered a putative regulators or co-factors of NFkB. Proceed with further characterization using tests of NFkB functions commonly known in the field.

11. If a given clone carries multiple inserts, perform “inverse” PCR* on both the untreated and puromycin-selected cells. Clone PCR products from the untreated cells in pCR2.1 cloning vector from Invitrogen (or any other vector that supports cloning of PCR products and allows for easy identification of colonies that carry inserts). It is essential that the total number of inserts identified via “inverse” PCR matches that of the distinct junction fragments identified by Southern blotting. Since the length of the junction fragment could not be readily predicted and very long fragments are poorly amplifiable by PCR, one may try several different enzymes or enzyme combinations for the initial step of the “inverse” PCR procedure. Identify the PCR products that were obtained from untreated, but not from the puromycin-selected cells**. Sequence such product(s) and find location of the targeted gene, as in step 10. Proceed with studies of putative regulators or co-factors of NFkB as in 10.

### EXAMPLE 3

### Genetic Screen for Factors Cooperating With the Loss of p53 in Skin Carcinogenesis

It is commonly accepted that more than a single genetic alteration has to occur within a single cell to enable tumor development. The actual tumor specimens usually contain great number of genetic alterations, including changes in hypermutable sites or rearrangements that involve dozens of genes. Hence, it is not trivial to unambiguously associate a specific genetic lesion with the oncogenic phenotype. Moreover, it is commonly accepted that multiple sets of such lesions may result in a similar pathological and clinical outcome, while the same lesion may have totally different consequences, depending on the genotype of a given cell (e.g. up-regulation of the same gene may have tumor-suppressive or tumor-promotive effects). Previous studies that embarked on comprehensive identification of oncogenes by retroviral insertional mutagenesis had to rely on analyzing hundreds or thousands of tumor samples and identifying the putative integration targets that occurred at somewhat higher than random frequency. These researchers then had to embark on a rather laborious projects to functionally correlate the product of the integration with the tumor phenotype. Our method has an additional bonus of unequivocally correlating a genetic event with transformed phenotype at the level of a single cell clone.

Although p53 is commonly altered in skin cancers, the loss of p53 is clearly insufficient for bona fide carcinogenesis of the skin, as indicated by frequent patches of p53-null cells that have very low probability of malignant progression. Additional events may enhance cell growth or cell survival. Oncogenesis may proceed through either loss of tumor suppressor functions or through activation of proto-oncogenes. Consequently, in an attempt to identify genetic alterations, which may cooperate with the loss of p53 in skin cancer progression, both loss-of-function and gain-of-function events are investigated. The identified genes may represent therapeutic targets or diagnostic markers for cancer treatment. Although the experiment is initially conducted on p53-deficient animals, the genes identified in this project should be tested in p53-positive cells/animals as well, to completely rule out the possibility of their p53-independent action. However, the events proven to be tumorigenic even in the presence of wild type p53, would still represent important findings.

1. By the methods commonly known in the art, establish ES cell lines that stably contain the following construct (See Figure).

2. Confirm transposition of the construct by expressing “Sleeping Beauty” transposase (SBT) and selecting for hygromycin-resistant clones. Use the ES cells that give the highest rate of transposition (at least 2 independent clones) to generate transgenic animal. Expression of β-galactosidase/hygromycin resistance fusion protein is restored upon excision of the transposon from its original location. Hence, the frequency of hygromycin-resistance cells after expression of SBT could be used as a measure of transposition efficiency at this stage. Alternatively, this could be done by performing assays of β-galactosidase activity in individual cells or total cultures by commonly used techniques. Multiple copies of the transgenic construct are preferable as long as they all retain the proper structure (e.g. as verified by Southern blotting and or PCR).

3. Cross the transgenic animals to animals lacking p53 tumor suppressor (commercially available). Preferably, both transposon-carrying and p53 deficient animals are of the same strain. If not, inbreeding should be used to achieve to high degree of homogeneity.

4. Generate a separate line of transgenic animals that express SBT in the skin in tetracycline—dependent manner. This line could be produced by first generating 2 separate lines (one with tetracycline regulator under the control of a keratinocyte-specific promoter and the other with SBT under a tetracycline-responsive promoter) and crossing them together. For tet-regulation, a variant of “tet-ON” system may be preferred, so that the animals express the SBT only when fed tetracycline. Establish mouse strains by continuous inbreeding.

5. Cross transgenic animals from #3 to the animals from #4. Establish mouse strains by continuous inbreeding. It is essential throughout the entire breeding phase of the project to maintain the maximal feasible number of transposons per genome. One way of doing so is to establish multiple syngeneic strains with small number of transposons and then breed them together. Diverse initial integration sites are important, since transposons have certain preference to relocate to physically proximal sites (this does not totally preclude integration at distant sites, albeit with a lower frequency).

6. Optimize conditions of transposition by varying the dose of tetracycline and conditions of treatment (e.g. mode or length of administration). Transposition could be monitored by appearance of β-gal positive cells. β-galactosidase activity is detected in tissue samples or individual cells using commonly used techniques. Transposition rates as high as 20% per transposon have been reported at least in some mammalian tissues. Having multiple copies of the transposon one may reasonably hope to improve per cell transposition rate to above 50%.

7. Induce the transposase by tetracycline treatment of the animals (topical treatment or feeding). Watch for the development of skin lesions.

8. Excise the cancerous lesions. Introduce the cells in tissue culture. Select cells where transposition has occurred using hygromycin (or sort them out using fluorescent-activated sorting with a fluorigeneic β-galactosidase substrate). This step should eliminate spontaneous tumors that have occurred without transposition and should also decrease the number of contaminating cells in transposition-induced tumors.

9. The integration event may be irrelevant to the tumorigenesis (“false positive”) or may play a role in cell growth or survival. (It is not uncommon for tumor cells to be hypersensitive to programmed cell death and require constant up-regulation of death-inhibiting factors to survive). Analyze the number of inserts per cell (transposons may be lost during transposition, so the total number of inserts may be lower than the number of transposons in the original genome) and compare the pattern to that in untreated tissues of the same animal. De novo integration sites found in the tumor, but not in the normal tissue of the animal (“differential sites”) are likely to indicate genes important for tumorigenesis.

10. Re-express SBT in the selected cells. Optionally, at this step, expression of SBT from the stably integrated construct may be augmented by transient expression (e.g. via transient transfection or from an appropriately engineered adenovirus).

11. If the integration at the “differential site” is essential for survival, one may expect that re-expression of SBT would be at least partially toxic and/or Southern blotting comparison of pre-treated and treated cells would reveal retention of the specific “differential site” essentially in all of the survivors. Recover the border fragments between the insert and the host DNA (e.g. via “inverse” PCR). Confirm by sequencing the fragments that the terminal repeats of the transposon are intact (that is, failure to show transposition among SBT-treated cells is not due to structural defect in the transposon). If this is the case, identify the gene at the integration site. If unknown, proceed with the functional characterization with conventional techniques. This gene is likely to play a role in cell death or survival.

12. If transposition has been achieved, expand individual cells post SBT treatment and graft such cells, as well as their untreated counterparts, onto a syngeneic animal or an immuno-deficient animal. Difference in the growth of tumors harboring or lacking the insert is the indication of the involvement of the said insert in tumorigenesis. Identify the integration site (via “inverse PCR”) and continue characterization of the gene at that site using conventional techniques.

Notes:

1. If transposition efficiency is high (e.g. >20% per copy of a transposon) one may opt for grafting onto a syngeneic animal multiple individual clones directly after re-expression of SBT (step 10) and then compare the pattern of integration sites in the clones that maintained or lost tumorigenicity. This should allow correlation between a given “differential site” and tumorigenic potential of a cell. In case of inserts, which are indispensable for survival, they would be the ones never found relocated as discussed at step 11.

2. One may identify the integration sites in the excised tumor and normal tissue via “inverse PCR”. Knowing the sequence at the “differential sites” should allow one to design PCR primers specific for the given locus. Consequently, one may use PCR with such primers to screen retention or loss the insert in individual SBT-treated clones (steps 11-12).

What is claimed is:

1. A promoter insertion construct for identifying a gene whose product modulates a specific control phenotype, said promoter insertion construct comprising:
a promoter element, a downstream recognition site for a disrupting agent having a site specific recombinase activity, and an upstream recognition site for the disrupting agent;
wherein the recognition sites are configured such that treatment of a DNA molecule comprising said promoter insertion construct and flanking downstream and upstream DNA sequences results in removal of the promoter from the DNA molecule; and
wherein said promoter insertion construct lacks a downstream termination sequence, a splice donor sequence, and a splice acceptor sequence.

2. The promoter insertion construct of claim 1, wherein the promoter insertion construct comprises
a) a promoterless marker gene that is downstream of and operably linked with the promoter element and an internal ribosome entry site downstream of the promoterless marker gene, or
b) a marker gene element that is upstream of the promoter element, said marker gene element comprising a marker gene, and a second promoter element for promoting expression of the marker gene product, or
c) both a and b.

3. The promoter insertion construct of claim 1, wherein the disrupting agent is a recombinase.

4. The promoter insertion construct of claim 1, wherein the disrupting agent is a transposase.

5. A method of identifying a gene whose product modulates a control phenotype of interest, comprising:
introducing the promoter insertion construct of claim 1 into the genomes of a collection of host cells having the control phenotype of interest to provide a population of mutagenized cells;
selecting mutagenized cells exhibiting a mutant phenotype of interest to provide a pool of mutant cells;
treating the mutant cells with a disrupting agent having recombinase activity;
detecting changes or the lack thereof in the linkage between the promoter element and downstream host genomic DNA sequences in treated, mutant cells, or in treated mutant cells and untreated, mutant cells;
correlating changes or lack thereof in the linkage between the promoter element and the downstream host genomic DNA sequences in treated mutant cells, or in both untreated and treated mutant cells, with the phenotypes of said cells;
wherein a host genomic DNA fragment that
a) is operably linked with the promoter element in untreated, mutant cells that display the mutant phenotype, but is not operably linked with the promoter element in treated cells that display the control phenotype, or
b) is operably linked with the promoter insertion construct in treated cells that maintain the mutant phenotype, but is not operably linked with the promoter insertion construct in treated cells that display the control phenotype, or
c) both a and b.
encodes a product that modulates the control phenotype of interest.

6. The method of claim 5, wherein the changes or lack thereof in the linkage between the promoter element and the downstream host genomic DNA sequences is detected by sequencing.

7. The method of claim 5, wherein the percentage of mutant cells that spontaneously revert to the control phenotype without treatment with the disrupting agent is compared to the percentage of mutant cells that revert to the control phenotype after treatment with the disrupting agent.

8. The method of claim 5, wherein the promoter insertion construct further comprises: one or more marker genes, and
wherein the method further comprises:
i) identifying mutagenized cells by monitoring expression of the marker gene;
ii) identifying treated, mutant cells in which the linkage between the promoter element and the downstream host genomic sequence has been disrupted by monitoring expression of the marker gene; and
iii) identifying treated, mutant cells in which the linkage between the promoter element and the downstream host genomic sequence has been maintained by monitoring expression of the marker gene.
iv) or any combination of steps i, ii, and iii.

9. The method of claim 5, wherein said host cells comprises a selection system comprising:
one or more constructs comprising a second promoter element and at least one marker gene that is operably linked with the second promoter element, wherein said second promoter element promotes expression of an RNA or protein that is associated with the control phenotype.

10. A promoter insertion construct for identifying a gene whose product modulates a control phenotype of interest, comprising:
a promoter element and a downstream recognition site for a disrupting agent having recombinase activity, wherein said construct lacks a termination sequence, a splice donor sequence, and a splice acceptor sequence, and
wherein the disrupting agent is a recombinase, and a circular double-stranded DNA molecule comprising
a recognition site for the recombinase, wherein said circular double-stranded DNA molecule lacks a promoter for promoting expression of the marker gene, a splice donor site, a splice acceptor site and a termination sequence.

11. The promoter insertion construct of claim 10, wherein said circular double-stranded DNA molecule further comprises a promoterless marker gene.

12. A method of identifying a gene whose product modulates a control phenotype of interest, comprising:
introducing the promoter insertion construct of claim 10 into the genomes of a collection of host cells having the control phenotype of interest to provide a population of mutagenized cells;
selecting mutagenized cells exhibiting a mutant phenotype to provide a pool of mutant cells;
treating the mutant cells with the disrupting agent;
detecting changes or the lack thereof in the linkage between the promoter element and downstream host genomic DNA sequences in treated mutant cells, or in both treated and untreated mutant cells;
correlating the changes or lack thereof in the linkage between the promoter element and the downstream host genomic DNA sequences in treated mutant cells, or in both untreated and treated mutant cells, with the phenotypes of said cells;
wherein a host genomic DNA fragment that
a) is operably linked with the promoter element in untreated, mutant cells that display the mutant phenotype, but is not operably linked with the promoter element in treated cells that display the control phenotype, or
b) is operably linked with the promoter insertion construct in treated cells that maintain the mutant phenotype, but is not operably linked with the promoter insertion construct in treated cells that display the control phenotype, or
c) both a and b.
encodes a product that modulates the control phenotype of interest.

13. The method of claim 12, wherein the percentage of mutant cells that spontaneously revert to the control phenotype without treatment with the disrupting agent is compared to the percentage of mutant cells that revert to the control phenotype after treatment with the disrupting agent.

14. The method of claim 12, wherein said host cells comprise a selection system comprising:
one or more constructs comprising a second promoter element and at least one marker gene that is operably linked with the second promoter element, wherein said second promoter element promotes expression of an RNA or protein that is associated with the control phenotype.

15. A promoter insertion construct for identifying a gene whose product modulates a specific control phenotype, said promoter insertion construct comprising:
a promoter element, a downstream recognition site for a disrupting agent having site specific recombinase activity, an upstream recognition site for the disrupting agent; and an RNA polyadenylation sequence or a transcription terminator sequence, or both upstream of the upstream recognition site;
wherein the recognition sites are configured such that treatment of a DNA molecule comprising said promoter insertion construct and flanking downstream and upstream DNA sequences results in inversion of the promoter within the DNA molecule; and
wherein said promoter insertion construct lacks a downstream termination sequence, a splice donor sequence, and a splice acceptor sequence.

16. The promoter insertion construct of claim 15, wherein the promoter insertion construct comprises
a) a promoterless marker gene that is downstream of and operably linked with the promoter element and an internal ribosome entry site downstream of the promoterless marker gene,
b) a promoterless marker gene that is upstream of the upstream recognition site and, optionally, an internal ribosome entry site upstream of the promoterless marker gene;
c) a marker gene element that is upstream of the promoter and downstream of the upstream recognition site, or
d) any combination of a, b, and c.

17. A method of identifying a gene whose product modulates a control phenotype of interest, comprising:
introducing the promoter insertion construct of claim 14 into the genomes of a collection of host cells having the control phenotype of interest to provide a population of mutagenized cells;
selecting mutagenized cells exhibiting a mutant phenotype to provide a pool of mutant cells;
treating the mutant cells with a disrupting agent having recombinase activity;
detecting changes or the lack thereof in the linkage between the promoter element and downstream host genomic DNA sequences in treated mutant cells, or in both treated and untreated mutant cells;
correlating the changes or lack thereof in the linkage between the promoter element and the downstream host genomic DNA sequences treated mutant cells, or in both untreated and treated mutant cells, with the phenotypes of said cells;
wherein a host genomic DNA fragment that
a) is operably linked with the promoter element in untreated, mutant cells that display the mutant phenotype, but is not operably linked with the promoter element in treated cells that display the control phenotype, or
b) is operably linked with the promoter insertion construct in treated cells that maintain the mutant phenotype, but is not operably linked with the promoter insertion construct in treated cells that display the control phenotype, or
c) both a and b.
encodes a product that modulates the control phenotype of interest.

18. The method of claim 17, wherein the promoter insertion construct further comprises one or more marker genes or promoterless marker genes, and
wherein the method further comprises:
i) identifying mutagenized cells by monitoring expression of the marker gene;
ii) identifying treated, mutant cells in which the linkage between the promoter element and the downstream host genomic sequence has been disrupted by monitoring expression of the marker gene; and
iii) identifying treated, mutant cells in which the linkage between the promoter element and the downstream host genomic sequence has been maintained by monitoring expression of the marker gene, or
iv) any combination of i, ii, and iii.

19. A promoter insertion construct for identifying a gene whose product modulates a specific control phenotype, said promoter expression construct comprising:
a promoter element, a downstream recognition site for a disrupting agent having recombinase activity, an upstream recognition site for the disrupting agent, a promoterless marker gene that is downstream of and operably linked to the promoter element; and an internal ribosome entry site that is downstream of the promoterless marker gene;
wherein said promoter insertion construct lacks a downstream termination site.

20. The promoter insertion construct of claim 19, wherein said construct comprises:
a) a translation start site, and
b) an uncoupled splice donor site.

21. The promoter insertion construct of claim 19, wherein the recognition sites are configured such that treatment of a DNA molecule comprising said promoter insertion construct and flanking downstream and upstream DNA sequences results in removal of the promoter from the DNA molecule

22. The promoter insertion construct of claim 18, wherein the construct comprises an RNA polyadenylation sequence, or a transcription terminator sequence, or both, upstream of the upstream recognition site; and
wherein the recognition sites are configured such that treatment of a DNA molecule comprising said promoter insertion construct and flanking downstream and upstream DNA sequences results in inversion of the promoter within the DNA molecule

23. A method of identifying a gene whose product modulates a control phenotype of interest, comprising:
introducing the promoter insertion construct of claim 18 into the genomes of a collection of host cells having the control phenotype of interest to provide a population of mutagenized cells;
selecting mutagenized cells exhibiting a mutant phenotype to provide a pool of mutant cells;
treating the mutant cells with a disrupting agent having recombinase activity;
detecting changes or the lack thereof in the linkage between the promoter element and downstream host genomic DNA sequences in treated mutant cells, or in both treated and untreated mutant cells;
correlating the changes or lack thereof in the linkage between the promoter element and the downstream host genomic DNA sequences in treated mutant cells, or in both untreated and treated mutant cells, with the phenotypes of said cells;
wherein a host genomic DNA fragment that
a) is operably linked with the promoter element in untreated, mutant cells that display the mutant phenotype, but is not operably linked with the promoter element in treated cells that display the control phenotype, or
b) is operably linked with the promoter insertion construct in treated cells that maintain the mutant phenotype, but is not operably linked with the promoter insertion construct in treated cells that display the control phenotype, or
c) both a and b.
encodes a product that modulates the control phenotype of interest.

24. The method of claim 23, wherein the disrupting agent is a recombinase.

25. The method of claim 23, wherein the disrupting agent is a transposase.

26. A promoter insertion construct set for identifying a gene whose product modulates a specific control phenotype, said promoter insertion construct set comprising three different promoter insertions constructs, wherein each of said three different promoter insertion constructs comprise:
a promoter element;
a first recognition site for a disrupting agent having recombinase activity, said first recognition site being upstream of the promoter element;
a second recognition site for the disrupting agent, said second recognition site being downstream of the promoter element;
an internal ribosome entry site downstream of the promoter element;
a translation start site downstream of the internal ribosome entry site;
an open reading frame sequence downstream of the translation start site and operably linked with the promoter element; and
an uncoupled splice donor site downstream of the of the open reading frame sequence;
wherein said promoter insertion construct lacks a downstream termination sequence and a splice acceptor sequence; and
wherein the uncoupled splice donor sites in the three different promoter insertion constructs are positioned in a different reading frame with respect to the translation start site.

27. The promoter insertion construct set of claim 26, wherein each of said constructs comprises a promoterless marker gene downstream and operably linked to the promoter element and an internal ribosome entry site downstream of the promoterless marker gene.

28. The promoter insertion construct set of claim 26, wherein each of said constructs is in a retrovirus-based vector or a transposon-based vector.

29. A method of identifying a gene whose product modulates a specific control phenotype, comprising:
introducing the promoter insertion construct set of claim 26 into the genomes of a collection of host cells having the control phenotype of interest to provide a population of mutagenized cells;
selecting mutagenized cells exhibiting a mutant phenotype to provide a pool of mutant cells;
treating the mutant cells with a disrupting agent having recombinase activity;
detecting changes or the lack thereof in the linkage between the promoter element and downstream host genomic DNA sequences in treated mutant cells, or in both treated and untreated mutant cells;
correlating changes or lack thereof in the linkage between the promoter element and the downstream host genomic DNA sequences in treated mutant cells, or in both untreated and treated mutant cells, with the phenotypes of said cells;
wherein a host genomic DNA fragment that
a) is operably linked with the promoter element in untreated, mutant cells that display the mutant phenotype, but is not operably linked with the promoter element in treated cells that display the control phenotype, or
b) is operably linked with the promoter insertion construct in treated cells that maintain the mutant phenotype, but is not operably linked with the promoter insertion construct in treated cells that display the control phenotype, or
c) both a and b.
encodes a product that modulates the control phenotype of interest.

30. The method of claim 29, wherein the promoter insertion construct further comprises one or more marker genes or promoterless marker genes, and
wherein the method further comprises:
i) identifying mutagenized cells by monitoring expression of the marker gene;
ii) identifying treated, mutant cells in which the linkage between the promoter element and the downstream host genomic sequence has been disrupted by monitoring expression of the marker gene;
iii) identifying treated, mutant cells in which the linkage between the promoter element and the downstream host genomic sequence has been maintained by monitoring expression of the marker gene; or
iv) any combination of a, b, and c.

31. A promoter insertion construct set for identifying a gene whose product modulates a control phenotype of interest, said set comprising three different promoter insertion constructs, each of said promoter insertion constructs, comprising:
a promoter element;
a downstream recognition site for a disrupting agent having recombinase activity;
an internal ribosome entry site downstream of the promoter element;
a translation start site downstream of the internal ribosome entry site;
an open reading frame sequence downstream of the translation start site and operably linked with the promoter element; and
an uncoupled splice donor site downstream of the of the open reading frame sequence;
wherein the uncoupled splice donor sites in the three different promoter insertion constructs are positioned in a different reading frame with respect to the translation start site;
wherein said construct lacks a termination sequence; and
wherein the disrupting agent is a recombinase, and a circular double-stranded DNA molecule comprising:
a recognition site for the recombinase.

32. The promoter insertion construct set of claim 31, wherein the plasmid comprises a promterless marker gene.

33. A method of identifying a gene whose product modulates a specific control phenotype, comprising:
introducing the promoter insertion construct set of claim 31 into the genomes of a collection of host cells having the control phenotype of interest to provide a population of mutagenized cells;
selecting mutagenized cells exhibiting a mutant phenotype to provide a pool of mutant cells;
treating the mutant cells with a disrupting agent having recombinase activity;
detecting changes or the lack thereof in the linkage between the promoter element and downstream host genomic DNA sequences in treated mutant cells, or in both untreated and treated mutant cells;
correlating the changes or lack thereof in the linkage between the promoter element and the downstream host genomic DNA sequences in treated mutant cells, or in both the untreated and treated mutant cells, with the phenotypes of said cells;
wherein a host genomic DNA fragment that
a) is operably linked with the promoter element in untreated, mutant cells that display the mutant phenotype, but is not operably linked with the promoter element in treated cells that display the control phenotype, or
b) is operably linked with the promoter insertion construct in treated cells that maintain the mutant phenotype, but is not operably linked with the promoter insertion construct in treated cells that display the control phenotype, or
c) both a and b.
encodes a product that modulates the control phenotype of interest.

34. The methods of claims 5, 12, 17, 23, 29, and 33, wherein multiple copies of the promoter insertion construct are inserted into the genome of each of the host cells.

35. A recombinant cell for identifying genes whose products modulate a select biological process, comprising:
the promoter insertion construct of claims 1, 10, 15, 19, or 26 wherein said promoter insertion construct is integrated into the cell's genome.

36. The recombinant cell of claim 35, wherein multiple copies of the promoter insertion construct are integrated into the cell's genome.

37. The recombinant cell of claim 35, wherein the cell comprises a selection system comprising:
one or more constructs comprising a second promoter element and at least one marker gene that is operably linked with the second promoter element, wherein said second promoter element promotes expression of an RNA or protein that is associated with the control phenotype.

38. The recombinant cell of claim 35 wherein the marker gene is a selectable marker gene.

39. The recombinant cell of claim 35, wherein the second promoter element is operably linked with a positive selectable marker gene, or a negative selectable marker gene, or both.
